(19) United States
(12) Patent Application Publication
(10) Pub. No.: US 2016/0158289 A1
(43) Pub. Date: Jun. 9, 2016

(54) MATERIALS AND METHODS FOR USING ADIPOSE STEM CELLS TO TREAT LUNG INJURY AND DISEASE

(71) Applicants: Keith Leonard March, Carmel, IN (US); Irina Petrache, Indianapolis, IN (US)

(72) Inventors: Keith Leonard March, Carmel, IN (US); Irina Petrache, Indianapolis, IN (US)

(21) Appl. No.: 15/041,781

(22) Filed: Feb. 11, 2016

Related U.S. Application Data
(63) Continuation of application No. 13/265,263, filed on Jan. 4, 2012.
(60) Provisional application No. 61/170,910, filed on Apr. 20, 2009.

(51) Int. Cl. A61K 35/28; C12N 5/0775

(52) U.S. Cl. CPC A61K 35/28; C12N 5/0667; C12N 2502/1382

(57) ABSTRACT

The present disclosure provides methods for treating patients with acute or chronic lung disease or injury to the lungs including injury caused by exposure to cigarette smoke other irritants or another cause of pulmonary distress. Typical conditions that can be treated include conditions that cause inflammation in the lung or the death of lung endothelial cells. Treatment of other conditions such as compromised bone marrow function and cachexia can also be treated by the inventive methods disclosed herein. These methods including contacting Adipose Stem Cells (ASC) or media conditioned by contact with ASC (ASC-CM) or various factors secreted by the same including the media or components of the media with lung tissue and cells. In some instances the ASC used is harvested from the patient's own adipose tissue while in other instances the source is an exogenous donor.

### PRIORITY CLAIM

This application is related to, claims the priority benefit of, and is a U.S. continuation application of, U.S. patent application Ser. No. 13/265,263, filed Jan. 4, 2012, which is related to, claims the priority benefit of, and is a U.S. national stage (§371) application of, International Application Serial No. PCT/US2010/031808, filed Apr. 20, 2010, which is related to, and claims the priority benefit of, U.S. Provisional Patent Application Ser. No. 61/170,910, filed Apr. 20, 2009, the entire contents of each of the foregoing applications incorporated herein by reference in their entirety.

### FIELD OF THE INVENTION

Aspects related to using Adipose Stem Cells (ASC) or various components of or derived from ASC or produced by ASC to treat lung disease or injury, including lung disease often times caused by exposure to smoke.

### BACKGROUND

Lung disease and lung injury resulting in compromised pulmonary function are debilitating and oftentimes fatal conditions These pathologies include, Adult Respiratory Distress Syndrome (ARDS), post-traumatic ARDS, lung transplant disease, Chronic Obstructive Pulmonary Disease (COPDs) including emphysema and chronic bronchitis, chronic obstructive bronchitis, allergies, pulmonary hypertension and the like. Symptoms of some, but not necessarily all of these pathologies may include inflammation, endothelial cell death and many of them are linked to or at least aggravated by cigarette smoking. Many of these disease and conditions are characterized by the progressive lose of lung tissue and function. For example, patients affected by emphysema often exhibit progressive respiratory symptoms including loss of lung function, which in many culminates in respiratory failure, as well as systemic symptoms such as weight loss, which may lead to cachexia.

Over 3.1 million Americans have been diagnosed with emphysema. Emphysema and chronic bronchitis are the two components of the syndrome referred to COPD which is the fourth leading cause of death in America. Pulmonary emphysema is a prevalent fatal disease, characterized by loss of both matrix and cellular elements of the lung, thus impairing gas exchange between the alveolar space and the capillary blood. Emphysema is defined as “a condition of the lung characterized by abnormal, permanent enlargement of airspaces distal to the terminal bronchiole, accompanied by destruction of their walls, with or without obvious fibrosis”. Report of a National Heart, Lung, and Blood Institute, Division of Lung Diseases workshop, Am Rev Respir Dis 132, 182-185. (1985). The concepts of permanent and destruction are critical in this definition as they convey the unique and characteristic distinguishing features of a disease process ultimately leading to the disappearance of lung tissue.

Because these conditions affect both life span and the quality of life for a large number of people these conditions are the focus of a large amount of research. And considerable progress has been made in understanding the causes of these pathologies. For example, while our understanding of the pathogenesis of emphysema has increased dramatically in the past decade there are still very few treatment options for this disease, and none of these treatments are curative. A truly effective treatment for these pathologies is one which will halt the loss of alveolar wall and even repair or reverse the damage that has already occurred. Unfortunately the standard of care for most patients with such a diagnosis consists of managing the symptoms of the disease or supporting lung function in hopes that the patient's innate ability to arrest and/or repair damage will improve the patient's well being. Given, the seriousness of these diseases and conditions and the lack of adequate treatments to from them there is a pressing need for effective therapies to treat chronic and acute lung injury and disease. Various aspects of the instant invention seek to address this need.

### SUMMARY

Some embodiments include methods for treating a patient presenting symptoms of acute or chronic lung injury or disease. Lung diseases that are readily treatable using these methods include, but are not limited to, lung diseases and injuries that involve inflammation and/or the premature death of endothelial cells. These conditions include, but are not limited to, Adult Respiratory Distress Syndrome (ARDS), post-traumatic ARDS, emphysema, Chronic Obstructive Pulmonary Distress syndrome (COPD), chronic bronchitis, pulmonary hypertension, or other pulmonary pathologies by administering a therapeutic dose of adult adipose stem cells (ASC) or a therapeutic dose of a molecular substance derived from ASCs such as specific factors secreted by ASC when they are cultured in vitro or the growth media itself that has been conditioned by the growth of ASC in the media. These cells or cellular products may be obtained from the patient to be treated or from an exogenous source such as a donor and they may be manipulated and/or modified to enhance their therapeutic function. ASCs or molecular substances directly derived from these cells may be administered via a variety of methods including systemically or by inhalation by the patient undergoing treatment.

Some aspects are directed to methods for treating patients with emphysema or COPD and more particularly to methods for treating a patient with emphysema or COPD by the means of infusing adult adipose tissue-derived stem cells or their products into a patient.

Some aspects of the present invention provide materials and/or methods for treating a patient having a lung disease or disorder such as one characterized by inflammation or cell death and tissue loss by administering a therapeutically effective amount of ASC or molecular substances directly derived from these cells, such as ASC growth media conditioned by the ASC as they grow in the media (ASC CM). The adult adipose stem cells or molecular substances directly derived from these cells may be administered systemically or by inhalation.

In some embodiments, the ASC or molecular substances directly derived from these ASC compound cultured in vitro are administered systemically, by for example, injection.

In still other embodiments, the ASC or molecular substances directly derived from ASC cultured in vitro are administered by inhalation.

In some embodiments ASC is obtained by liposuction from the fat tissue of mammals including humans. In some embodiments these cells may be used by themselves or modified by molecular means to have a more effective function. In still other embodiments the molecular substances derived from these cells include, but not limited to, vascular growth factors, antiapoptotic factors, etc that are released from the adult adipose stem cells when they are grown outside the body in culture conditions and may be used to treat conditions such as lung damage or conditions such as cachexia or conditions that include a reduction in the production of progenitor cells by the bone marrow (BM). Similarly. ASC can be used to treat cachexia or conditions that include a loss of or reduction in of bone marrow function.

Some embodiments include protocols for administering ASC or ASC-CM that are similar to the protocols for administering of any other agent typically administered to a patient to treat a lung disease or injury. For example, ASC or ASC-may be administered by inhalation or by injection.

Some embodiments include methods for treating lung diseases and disorders comprising the steps of provide a therapeutic dose of ASC or ASC conditioned media i.e. in vitro growth or maintenance media that has been conditioned by contact with ASC (ASC-CM); identifying a patient who has been diagnosed with at least one respiratory condition and administering at least one therapeutic dose ASC or ASC-CM to the patient. In some embodiments the therapeutic dose includes between about 1.0×10⁵ ASC per kg⁻¹ of body weight to about 1.0×10⁸ ASC kg^{z} of body weight. In other embodiments the therapeutic dose includes between about 3.0×10⁵ ASC per kg⁻¹ of body weight to about 3.0×10⁷ ASC kg⁻¹ of body weight. And in still other embodiments the therapeutic dose includes about 1.0×10⁻⁵ ACS cells. In some embodiments the patient suffers from at least one respiratory condition selected from the group consisting of, but not limited to the group consisting of: Adult Respiratory Distress Syndrome, post-traumatic Adult Respiratory Distress Syndrome, transplant lung disease, Chronic Obstructive Pulmonary Disease, emphysema, chronic obstructive bronchitis, bronchitis, an allergic reaction, damage due to bacterial or viral pneumonia, chronic asthma; exposure to irritants. In still other embodiments the patient may be diagnosed with pulmonary hypertension. In some embodiments the patient treated with the inventive methods presents inflammation of the lungs and/or the loss of endothelial cells through cellular death.

In some embodiments ASC used to treat the patient or is harvested from the patient or from a donor other than the patient. In some embodiments ASC-CM used to treat the patient or is made by contacting ASC growth media with ASC harvested from the patient or from a donor other than the patient. In some embodiments ASC cells are harvested for a human or animal and grown in vitro before being used in the inventive treatments. In still other embodiments a formulation of ASC is created by enriching a sample in ASC and this formulation is used without growing the ASC in vitro. In still other embodiments ASC harvested from an animal is then grown in vitro to increase the number of cells. Therapeutic doses of either ASC or ASC CM may be administered by at least one technique selected from the group consisting of: inhalation, ingestion and injection.

Other aspects of the invention include methods of treating patient diagnosed with or at risk for developing unwanted, pathological weight loss such as cachexia, these methods include providing at least one therapeutic dose of a composition selected from the group consisting of: ACS and ACS-CM; identifying a patient, wherein the patient has a diagnosis of pathologic weight loss or is at risk for pathologic weight loss; and administering said at least one therapeutic dose of the composition to the patient. In some embodiments the therapeutic dose of ACS is between about 1.0×10⁵ ASC kg⁻¹ of body weight to about 0.0×10⁸ ASC kg⁻¹ of body weight. While other embodiment the therapeutic dose of ACS is between about 3.0×10⁵ ASC per kg⁻¹ of body weight to about 3.0×10⁷ ASC per kg⁻¹ of body weight. In still other embodiments the therapeutic dose of ACS-CM for therapeutic use is created by contacting ASC growth media or maintenance media in vitro with ASC for between about 1 to about 7 days. Some embodiments include the further step of concentrating the ASC-CM at least 100 fold before using it a therapeutic setting. Concentration may be accomplished by any means commonly used in the art that does not significantly reduce the therapeutic effectiveness of the formulation including, for example, filtration.

In some embodiments the patient treated for weight loss by the inventive methods suffers from at least one respiratory condition selected from the group consisting of: Adult Respiratory Distress Syndrome, post-traumatic Adult Respiratory Distress Syndrome, transplant lung disease. Chronic Obstructive Pulmonary Disease, emphysema, chronic obstructive bronchitis, bronchitis, an allergic reaction, damage due to bacterial or viral pneumonia, chronic asthma; exposure to irritants. In still other embodiments the patient has a diagnosis of pulmonary hypertension. In some other embodiments the patient may be diagnosed with any condition that causes inflammation in the lung and/or the premature death of lung endothelial cells and/or the loss of lung tissue. In some embodiments the patient may be diagnosed with cachexia due to at least one of the following: disease, chemical poisoning, radiation poisoning, chemotherapy, anemia, and aging. In some embodiments the ASC is harvested from humans while in other embodiments it is arvested from other mammals. The ACS may be harvested from the patient being treated or form a donor other than the patient the patient undergoing treatment may be a human or another mammal. At least one therapeutic dose may be administered by any method known in the art including, but not limited to, inhalation, ingestion or injection.

Still another embodiment of the invention includes material or methods for stimulating the production of bone marrow derived progenitor cells, comprising the steps of: identifying a patient who has is diagnosed with reduced bone marrow function or is at risk for developing reduced bone marrow function; providing a therapeutic dose of a composition selected from the group consisting of: ASC and ASC-CM; and administering a therapeutic dose of the composition to the patient. In some embodiments the therapeutic dose of ACS is between about 1.0×10⁵ ASC per kg⁻¹ of body weight to about 1.0×10⁸ ASC per kg⁻¹ of body weight. While in still other embodiments the therapeutic dose of ACS is between about 1.0×10⁵ ASC per kg⁻¹ of body weight to about 1.0×10⁸ ASC per kg⁻¹ of body weight. And in still other embodiments the therapeutic dose is about 1×10⁵ ASC. In still other embodiments the therapeutic dose of ACS-CM for therapeutic use is created by contacting ASC growth or maintenance media in vitro with ASC for between about 1 to about 7 days. Some embodiments include the further step of concentrating the ASC-CM at least 100 fold before using it a therapeutic setting. Concentration may be accomplished by any means commonly used in the art that does not significantly reduce the therapeutic effectiveness of the formulation including, for example, filtration.

In some embodiments the patient suffering from a reduction in progenitor cell formation in the bone marrow is suffering from at least one respiratory condition that may include inflammation or the premature death of endothelial cells and/or the loss of lung tissue. In some embodiments the patient is suffering from at least one disease or condition selected from the group consisting of: Adult Respiratory Distress Syndrome, post-traumatic Adult Respiratory Distress Syndrome, transplant lung disease, Chronic Obstructive Pulmonary Disease, emphysema, chronic obstructive bronchitis, bronchitis, an allergic reaction, damage due to bacterial or viral pneumonia, chronic asthma; exposure to irritants. In some embodiments the patient is suffering from pulmonary hypertension. In some embodiments the patient is diagnosed with cachexia due to at least one of the following: disease, chemical poisoning, radiation poisoning, chemotherapy, anemia, and aging.

In some embodiments the ASC is harvested from humans while in other embodiments it is harvested from other mammals. The ACS may be harvested from the patient being treated or from a donor other than the patient. The patient undergoing treatment may be a human or another mammal. Therapeutic doses may be administered by any method known in the art including, but not limited to, inhalation, ingestion or injection.

### BRIEF DESCRIPTION OF THE FIGURES

FIG. 1A. Photomicrographs of murine lung tissue stained with X-Gal to detect the presence of ASC.

FIG. 1B. Photomicrographs of murine lung tissue probed with anti-GFP antibody to detect the presence of ASC in the tissue.

FIG. 1C. A graph showing the relative levels of GFP expression in murine lung homogenates made from the lungs of mice exposed to air, and cigarette smoke (CS) for two weeks followed by treatment with ASC and then sampled either 1 day or 7 days after treatment.

FIG. 2A. Graph showing alveolar macrophage levels measured in murine lungs contacted with Air, CS and CS+ASC.

FIG. 2B. Graph showing PMN/ml BAL levels measured in murine lungs contacted with Air, CS and CS+ASC.

FIG. 3. Photomicrographs of murine lung tissue stained to detect Caspase-3 IHC activity

FIG. 4A. Graph of Caspase-3 activity measured in control, control+human ASC, VEGFR inhibitor and VEGFR inhibitor+human ASC.

FIG. 4B. Graph of mean linear intercept of Caspase-3 activity determined for control, VEGFR inhibitor and VEGFR inhibitor+human ASC.

FIG. 5. Graph of Caspase-3 activity measured in samples from murine lung contacted with air, CS and CS+ASC.

FIG. 6. Western blots of lung homogenates harvested from lungs that were exposed to one of the following conditions: air (control); CS; air+ASC: and CS+ASC. The blot was probed with antibody to vincullin, phospho-Akt, Phospho-ERK1/2 and phospho-JNK.

FIG. 7A. Graph of Phosphorylated-p38 MAPK/total P-p38 MAPK measured in homogenates made from murine lung exposed to Air, CS and CS+ASC.

FIG. 7B. Graph of Phosphorylated-JNK/total JNK measured in homogenates made from murine lung exposed to Air, CS and CS+ASC.

FIG. 7C. Graph of total Phosphorylatcd-Akt/total Akt measured in homogenates made from murine lung exposed to Air, CS and CS+ASC.

FIG. 8A. Photomicrographs of murine Alveolar stained with hematoxyllin/eosin tissue harvested from lungs exposed to air. CS and CS+ASC.

FIG. 8A. Graph of Alveolar Surface Area measured in lungs of mice exposed to air, CS and CS+ASC.

FIG. 8C. Graph of lung volume measured in lungs of mice exposed to air, CS and CS+ASC.

FIG. 9. Graph illustrating the relative number of three different types of bone marrow derived progenitor cells: colony forming granulocytes, monocyte (CFU-GM); burst-forming-unit-erythroid (BFU-E); and colony forming unit granulocytes, moncytes and megakaryocyte (CFU-GEMM) measured in mice exposed to Air, CS and exposed to CS and treated with ASC.

FIG. 10A. Graph of results from wounding experiments conducted in vitro on a confluent monolayer of human lung microvascular endothelial cells. These results are from cells exposed to the following conditions: a control (Ctl); cells treated with adult human Adipose Stem Cell Conditioned Media (ASC-CM) no CS; control cells exposed to CS and (ASC-CM) exposed to CS.

FIG. 10B. Plot of Electrical Resistance (Ohms) versus time measured in vitro on a confluent monolayer of human lung microvascular endothelial cells after wounding. Three sets of cells were exposed to three different conditions, Control (Ctl) cells in standard media, ASC-CM and Fetal Bovine Serum and Conditional Media (FBS-CM).

FIG. 10C. Plot of Electrical Resistance (Ohms) measured versus time in vitro on a confluent monolayer of human lung microvascular endothelial cells that were exposed to cigarette smoke extract (CSE) after wounding. Three sets of cells were exposed to three different conditions, Control (Ctl) cells in standard media. ASC-CM and FBS-CM.

FIG. 11A. Plot of mouse weight measured in mice exposed to air, CS and CS+treatment with ASC.

FIG. 11B. Plot of mouse area of fat (mm⁻²) measured in mice exposed to air, CS and CS+treatment with ASC.

FIG. 11C. Photograph of dissections of mice showing fat stores in mice that were exposed to air, CS and CS+treatment with ASC.

FIG. 12. Photograph of mice showing their girth, three mice were photographed they were exposed to air, CS and CS+treatment with ASC, respectively.

### DESCRIPTION

For the purposes of promoting an understanding of the principles of the novel technology, reference will now be made to the preferred embodiments thereof, and specific language will be used to describe the same. It will nevertheless be understood that no limitation of the scope of the novel technology is thereby intended, such alterations, modifications, and further applications of the principles of the novel technology being contemplated as would normally occur to one skilled in the art to which the novel technology relates.

Any of the protocols, formulations, routes of administration and the like that have previously been used in the treatment of lung disorders may readily be modified for the practice of the present invention. In some cases, mechanical ventilation is appropriate. Such ventilation may include high-frequency oscillatory ventilation (HFOV) or other unconventional forms of mechanical ventilation. Theoretically, partial liquid ventilation (PLV) offers the advantage of lung lavage combined with ventilator support.

Therapeutic or otherwise efficacious dosages may be determined using an animal model, such as exposure to CS and other such models. These CS based models may be modified and adapted for use in various mammals including humans. The total dose of therapeutic agent may be administered in multiple doses or in a single dose. In certain embodiments, the compositions are administered alone, in other embodiments the compositions are administered in conjunction with other therapeutics directed to the pathology or directed to symptoms thereof.

As used herein, ‘ASC’ is an acronym for Adult Adipose Stem Cells used interchangeably with the term Adult Stein Cells these terms refer to the cell type and not to the age of the animal or human from which they were obtained.

As used herein, the terms ‘ASC-CM’ and ‘ASC CM’ (Adult Adipose Stem Cell Conditioned Media) are used interchangeably refer to media that was conditioned by in vitro exposure to ASC.

Appropriate dosages may be ascertained through the use of established assays for determining blood levels in conjunction with relevant dose response data. The final dosage regimen will be determined by the attending physician, considering factors which modify the action of drugs, e.g., the drug's specific activity, severity of the damage and the responsiveness of the patient, the age, condition, bodyweight, sex and diet of the patient, the severity of any infection, time of administration and other clinical factors. As studies are conducted, further information will emerge regarding appropriate dosage levels and duration of treatment for specific diseases and conditions.

Known methods and ready modifications of known methods formulating ASC, other and molecular substances derived from ASC may be used to practice the invention.

Unless specified otherwise the term ‘about’ means plus or minus 10 percent e.g. about 1.0 encompasses the range of 0.9 to 1.1.

Administration of these compositions according to the present invention may be accomplished by any route so long as access to the target cells, tissue or organ is accessible via the route used. In some instances the cells and other cellular products or derivatives thereof are formulated for local administration, such as by inhalantion. However, other conventional routes of administration, e.g., by subcutaneous, intravenous, intradermal, intramuscular, intramammary, intraperitoneal, intrapleural, intrathecal, intraocular, retrobulbar, intrapulmonary (e.g., long-term release), aerosol, sublingual, nasal, anal, vaginal, or transdermal delivery, or by surgical implantation at a particular site also is used particularly when oral administration is problematic. The treatment may consist of a single dose or a plurality of doses that are administered over a period of time.

Various aspects of the present invention can be employed in a wide variety of pharmaceutical forms; the compound can be employed neat or admixed with a pharmaceutically acceptable carrier or other excipients or additives. Generally speaking, the compound will be administered by inhalation, orally, locally, or intravenously. It will be appreciated that therapeutically acceptable salts of the compounds of the present invention may also be employed. The selection of dosage, rate/frequency and means of administration is well within the skill of the artisan and may be left to the judgment of the treating physician. The method of the present invention may be employed alone or in conjunction with other therapeutic regimens.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution is suitably buffered if necessary and the liquid diluent first rendered isotonic with sufficient saline or glucose. These particular aqueous solutions are especially suitable for intravenous, intramuscular, subcutaneous and intraperitoncal administration.

The frequency of dosing will depend on the pharmacokinetic parameters of the agents and the routes of administration. The optimal pharmaceutical formulation will be determined by one of skill in the art depending on the route of administration and the desired dosage. Such formulations may influence the physical state, stability, rate of in vivo release and rate of in vivo clearance of the administered agents. Depending on the route of administration a suitable dose may be calculated according to body weight, body surface areas or organ size. The availability of animal models is particularly useful in facilitating a determination of appropriate dosages of a given therapeutic formulation. Further refinement of the calculations necessary to determine the appropriate treatment dose is routinely made by those of ordinary skill in the art without undue experimentation, especially in light of the dosage information and assays disclosed herein as well as the pharmacokinetic data observed in animals or human clinical trials.

Research suggests that exaggerated capillary endothelial cell apoptosis, which may occur in the context of a vascular endothelial growth factor (VEGF)-deprived environment (1), is at least one mechanism contributing to lung injury in emphysema and this putative mechanism has become an important therapeutic target (1-4). Some studies have shown that adult mesenchymal precursor/stem cells of adipose tissue origin protect against apoptosis of endothelial cells from systemic vascular beds (5, 6). As disclosed herein, the ability of adipose-derived stem cells (ASC) to inhibit the apoptosis of lung endothelial cells in vivo and limit the lung injury induced by cigarette smoke (CS) was investigated.

Bone marrow (BM)-derived stem cells transplanted to the lungs can exhibit phenotypic and can acquire functional markers of airway or alveolar epithelial cells, interstitial cells and vascular endothelial cells (7). Potential lung protective and regenerative activities of both endothelial progenitor cells activated by the hepatocyte growth factor (HGF) and autologous ASC have been suggested in previous reports using an elastase-induced emphysema model (7, 8). Still, little has been reported in the context of using the cigarette smoke (CS) to induce lung disease and/or injury and to examine the regenerative potential of human or murine ASC in this for more relevant model for lung disease and injury. ASC constitutes a distinct progenitor cell population within the adipose stromal compartment that has the practical advantage of being available from a readily accessible and ethically uncontested source. For example, ACS can be obtained in large numbers via liposuction from adult animals or humans. And the subcutaneous adipose tissue contains pluripotent cells in the stromal (non-adipose) compartment that can differentiate into multiple cell lineages, including neurons, skeletal myocytes, osteoblasts, chondroblasts, adipocytes, and vascular wall cells (9). Previous studies demonstrated that the protective properties of ASC are at least in part attributable to their capability to secrete multiple pro-angiogenic and anti-apoptotic growth factors, including VEGF and HGF (10, 11), which act in a paracrine manner (11-14). In addition. ASC may directly partner with vascular endothelial cells to form vascular networks via a process of adult vasculogenesis (15).

As disclosed herein ASC can home to regions of pulmonary endothelial injury and promote endothelial integrity either by secreting anti-apoptotic factors and/or by directly supporting pulmonary endothelium as mural cells. Two established experimental models of CS exposure were used to test the efficacy of this therapeutic approach, including VEGF receptor (VEGFR) blockade-induced emphysema, which share with human emphysema characteristics such as alveolar apoptosis, oxidative stress, and alveolar space enlargement and destruction (3, 16, 17).

In addition to damaging pulmonary structures and function, long-term exposure to CS triggers clinically important extra-pulmonary manifestations, including cardiovascular disease (18, 19), total body weight loss (20, 21), and decreased bone-marrow derived stem cell differentiation and migration potential (22, 23). While there has been significant progress in understanding the pathogenesis of and developing therapies for the CS-induced cardiovascular dysfunction, much less is known about the mechanisms by which CS affects body mass and bone marrow (BM) function, and no treatments exist for these conditions.

As disclosed herein intravenous administration of adult ASC of either human or mouse origin resulted in the repair of small vessel injuries induced by CS or VEGFR inhibition. This therapeutic approach improved both the pulmonary and systemic health of animals tested using the CS model for lung injury and disease. These results provide a potent therapeutic option for treating acute and chronic lung injury and disease including, but not necessarily limited to COPD and other diseases involving disruption of the pulmonary architecture.

Although the environmental inducers in susceptible individuals have been identified, the mechanisms by which these initiate a loss of alveoli leading to emphysema are poorly understood. Over the past decades, inflammation and a protease/antiprotease imbalance have been proposed to act as downstream effectors of the lung destruction following chronic cigarette smoking, which accounts for most cases of emphysema. Pro-inflammatory stimuli are postulated to recruit and activate lung inflammatory cells, triggering matrix protease release and lung remodeling (47). However, these models fail to fully account for the mechanisms behind the eradication of septal structures and the unique nature of lung destruction as compared to alterations seen in other inflammatory lung diseases. To account for the permanent destruction seen in emphysema, excessive apoptosis of structural alveolar cells have emerged as a second major cause of the damage done to lungs in patients with emphysema. Excessive alveolar endothelial apoptosis is thought to cause capillary regression, with subsequent loss of alveolar wall (26).

Adipose stem cells or their products may be used to enhance the survival and the repair of cells in the lung that lead to treatment of emphysema or COPD or pulmonary hypertension. These conditions are characterized by abnormal loss of, or function of, endothelial cells in the lung. As disclosed herein adipose stem cells (ASC) when administered locally or systemically home or are trapped in the lung very efficiently. ASC themselves and/or factors derived from them enhance the survival and may participate in or even assume the normal function of resident cells in the lung, such as, but not limited to, lung endothelial or epithelial cells.

The present invention provides methods for treating a patient with emphysema or COPD comprising treating with a therapeutically effective amount of adult adipose stem cells or molecular substances directly derived from these cells. Adult adipose stem cells (ASC) are obtained from adipose tissues by techniques that include, but are not limited to, lipoaspiration and lipoexcision; preparations that include these cells may be prepared for administration either shortly following isolation, or after storage, culture, or other treatments of the cells.

As reported herein both murine and human ASC are capable of significantly ameliorating the pulmonary damage caused by CS exposure, even when administered mid-way during a temporally protracted CS exposure. The observed profound anti-apoptotic effects of ASC in the murine lung as well as the vascular protective properties of paracrine factors secreted by these cells render such therapy a potentially promising intervention in emphysema. Recognition of the importance of endothelial apoptosis in experimental pulmonary emphysema (1, 3, 4), including following tobacco smoke exposure (36, 37), has prompted a focus on the potential role for vascular cell-responsive growth factor modulation as a novel approach to treatment. Models involving endothelial apoptosis caused by either exposure to CS or specific impairment of endothelial survival by VEGFR-blockade allow the evaluation of putative therapies in the context of a clinically relevant toxic exposure and a specific molecular lesion, respectively. As previously reported, ASC can elicit both angiogenic and anti-apoptotic effects in multiple systems (5, 6, 14). Prior studies have also shown that both intravenous systemic administration of ASC and local placement of ASC on a synthetic scaffold could limit the extent of elastase-induced emphysema and accelerate lung growth after experimental lung volume reduction surgery in rats (38-40). While these reports describe a role for ASC in artificial elastase modeled injury they do not appear to disclose the efficacy of using ASC to treat the damage done to lungs by exposure to cigarette smoke.

The therapeutic effects of ASC on the pulmonary system may engage multiple mechanisms, including secretion of anti-apoptotic factors with paracrine protective action on neighboring resident lung cells, activation of endogenous progenitor cell cycling and differentiation, rescue and recruitment of circulating cells engaged in pulmonary repair, and direct differentiation into pulmonary epithelial cells. The relatively low number of ASC detectable in lung tissue several days following administration, coupled with their effective anti-apoptotic and overall lung protective effects, suggest that an important therapeutic function of ASC may be to promote endogenous repair processes and limit damage through paracrine effects. Similar protective effects of ASC delivery in the VEGF-inhibition model of emphysema supports the notion that VEGF is one of the factors secreted by ASC which exert protective effects on lung endothelial cells. Those types of effects have been reported in the context of cultured endothelial cells (10). However, since ASC continued to be detected at latter timepoints intercalated among epithelial cells remains a distinct possibility. It is possible that ASC may be directly participating in tissue regeneration to limit CS-induced lung injury.

Reported herein is that adult ASC promote the repair of the lung endothelial barrier function, even in the presence of CS. While unexpected these vascular protective properties of ASC are in agreement with previous reports that bone-marrow derived progenitor stem cells that can reduce lung vascular permeability (41) and may be explained by their endogenous localization in the adipose tissue in a perivascular niche, where they exhibit pre-pericytes markers (24). Furthermore, ASC secrete potent pro-survival factors and ACS-conditional media (ASC-CM) may exert an anti-apoptotic effect on systemic vascular endothelial cells. Reports in the literature have focused on actions HGF and VEGF on angiogenesis and the formation of new vessels (33).

Remarkably, the marked effects of chronic CS exposure on body weight, adipose depots, and hematopoietic progenitor cycling and colony formation of multiple Bone Marrow (BM) colony-forming types were substantially reversed by ASC, demonstrating that the provision of ASC results in systemic protection against diverse pathologies induced by such smoke exposure. Substantial weight loss in the context of cigarette smoking is a well-known clinical phenomenon and described previously in C57/Bl6 mice exposed to CS for 9 weeks (20, 21), a similar effect of CS in the DBA/2J mice was also noted. The weight loss (cachexia) associated with advanced stages of COPD portends a poor prognosis for these patients, even after smoking cessation, and has no effective treatment. Therefore, the ability of the ASC to reverse pathologic weight loss may be of great therapeutic promise. Without being bound by any theory, hypothesis or specific explanation, cachexia may be the result of excessive circulating TNF levels (42). In fact a recent study of BM-derived mesenchymal stem cells (BM-MSC), which bear substantial similarity to ASC (43, 44), demonstrated that BM-MSC, which also predominantly localized in the lung following intravenous administration, promote systemic tissue repair by secreting several specific molecules in response to elevated levels of circulating TNF found in the context of tissue damage (45). It is intriguing to speculate that such a TNF mediated activation of ASC may likewise induce secretion of a spectrum of molecules that block the cachectic effects of TNF.

Bone marrow (BM) is the key adult repository for hematopoietic stem cells and endothelial progenitors, and each of these populations has been reported to be depressed due to CS or nicotine, a major component of CS (22, 23, 32). In addition, reports by Liu et al, among others, have noted that CS causes the release of immature eosinophils from BM and that Balb/c mice exposed to nicotine demonstrate impairment of hematopoetic stem cell migration, which is hypothesized to alter stem cell homing (31, 32, 46). Further in vitro data have demonstrated that CS extract strikingly diminishes BM progenitor cell chemotaxis in Boyden chamber assays. As disclosed herein, analysis of the BM from mice exposed to CS revealed that BM-derived progenitor cells had diminished proliferation capacity and were decreased in number, suggesting that decreased circulation of hematopoietic progenitor cells due to CS exposure may contribute to the body's inability to repair the pulmonary tissue in COPD. Thus, ASC-induced restoration of BM progenitor cell cycling and numbers may constitute, a heretofor uncovered mechanism by which these cells exert vascular protective effects. The mechanism by which administration of ASC restored the proliferation of the hematopoietic progenitor cells remains unknown, but could potentially involve molecules that overlap with those active in sustaining body mass as described above.

In conclusion, adult ASC exert protective and reparative properties against lung endothelial injury and against pulmonary and systemic deleterious effects of CS exposure, including airspace enlargement, weight loss, and BM suppression. These cells, which are a readily available population of highly proliferative and clonogenic cells resident in the stromal fraction of adipose tissues and may be readily expanded in vitro may represent a potential therapeutic option in lung diseases characterized by excessive apoptosis, including pulmonary emphysema.

### EXAMPLES

### Material and Methods

### Reagents and Antibodies.

All chemical reagents were purchased from Sigma-Aldrich (St. Louis, Mo.), unless otherwise stated.

### ASC Harvesting, Characterization, and Culture.

Human ASC were isolated from human subcutaneous adipose tissue samples obtained from liposuction procedures as previously described (24). Briefly, samples were digested in collagenase Type I solution (Worthington Biochemical. Lakewood, N.J.) under agitation for 2 hours at 37° C., centrifuged at 300 g for 8 minutes to separate the stromal cell fraction (pellet) from adipocytes. The pellets were filtered through 250 μm Nitex filters (Sefar America Inc., Kansas City. Mo.) and treated with red cell lysis buffer (154 mM NH₄Cl₂, 10 mM KHCO₃, and 0.1 mM EDTA). The final pellet was re-suspended and cultured in EGM-2mv (Lonza). ASC were passaged when 60-80% confluent and used at passage 3-6. Purity of ASC samples from endothelial cell contamination was confirmed by staining ASC monolayers with anti-CD31 antibodies. Mouse ASC were isolated in a similar fashion from adult DBA/2J, B6.129P2-Apoe^{tm1Unc}/J (Apo E), and B6; 129S-Gt(ROSA)26Sor/J (“ROSA26”) mice (25). Morbidity or mortality from embolic lodging of ASC administration was not seen unless the number of ASC injected exceeded 5×10⁵, or the passage number of ASC expanded ex vivo exceeded 3 when a larger cellular size was noted, which prompted us to utilize mouse ASC up to passage 3, followed by filtration through a 40 μM filter prior to injection.

### Animal Studies.

Animal studies were approved by the Animal Care and Use Committee of Indiana University. C57Bl/6, ApoE, ROSA26, and DBA/2J mice were from Jackson Labs. At the end of experiments, the mice were euthanized and the tissue was processed as described (3). In addition, mice underwent bronchoalveolar lavage (BAL), utilizing PBS (0.6 ml). BAL cells were sedimented via centrifugation and counted after Giemsa staining of cytospins. The remaining acellular fluid was then snap-frozen in liquid nitrogen and stored at −80° C. for further analysis.

### In Vivo CS Exposure.

Exposure to CS was performed as previously described [40]. C57Bl/6 (female, age 12 weeks; n=5-10 per group) or DBA/2J (male; age 12-14 weeks; n=5-10 per group) mice were exposed to CS or ambient air for up to 24 weeks. Briefly, mice were exposed to 11% mainstream and 89% side-stream smoke from reference cigarettes (3R4F; Tobacco Research Institute, Kentucky) using a Teague 10E whole body exposure apparatus (Teague Enterprise, CA). The exposure chamber atmosphere was monitored for total suspended particulates (average 90 mg/m³) and carbon monoxide (average 350 ppm). In all CS experiments, mice were euthanized and lungs were processed as previously described (3) the day following the last day of CS exposure.

### Blockade of the VEGF Receptor.

Access to the VEGF receptor was blocked using previously described methods (3). NOD.Cg-Prkdc^{scid} IL2Rγ^{null} (NS2) mice (Indiana University Cancer Center Stem Cell Core) (female; age 9 weeks;) were injected with SU5416 (Calbiochem; 20 mg/kg, subcutaneously) or vehicle, carboxymcthylcellulose (CMC) and the mice were euthanized at the indicated time.

### Lung Disintegration and ASC Detection by Flow Cytometry:

Following euthanasia, the mouse trachea was cannulated and the thoracic cavity was opened. The lung vasculature was perfused with sterile PBS (20 ml; Invitrogen). The lung tissue was digested in 10% FBS in DMEM, 6.5 g/ml DNAse I, and 12 μg/ml Collagenase I (Roche) (30 min; shaking 200 rpm; 37° C.). The cell suspension was strained through a 70 μm cell strainer (Fisher Scientific) and cells were collected by centrifugation (500×g; 5 minutes; 4° C.). Cells were resuspended in Geyes solution, centrifuged as before, and collected in PBS, followed by fixation with paraformaldehyde (1%; 30 minutes; 21° C.). Cells were then collected by centrifugation (500×g; 5 min; 21° C.), and resuspended in PBS for flow cytometry. Thirty thousand cells were analyzed for the presence of Vybrant DiI (Molecular Probes V22885) using flow cytometry (FC 500; emission 575 nm, excitation 488 nm).

### Apoptosis Measurements.

Apoptosis was detected in inflated fixed lung sections, enabling specific evaluation of alveoli, rather than large airways and vessels (26), via active caspase-3 IHC (Abcam and Cell Signaling) (3), using rat serum as negative control. The immunostaining for active caspase-3 was followed by DAPI (Molecular Probes) nuclear counter-staining. Executioner caspase (caspase-3 and/or -7) activity was measured with ApoONE homogeneous Caspase-3/7 assay kit (Promega, Madison, Wis.) as described (3). Human recombinant caspase-3 (Calbiochem) was utilized as positive control.

### Immunohistochemistry (IHC).

Paraffin sections, or for some applications, (GFP visualization) cryosections were blocked with 10% rabbit (or goat serum, if secondary antibody from goat) and incubated with primary antibodies or control antibodies. Anti-caspase-3 (Cell Signaling) antibody was incubated for 1 hour at room temperature or at 4° C. overnight. Bound antibody was detected according to the manufacturer's instructions using a biotin-conjugated goat anti-rat IgG secondary antibody (Vector Laboratories, Burlingame, Calif.; 1:100) and Streptavidin-coupled phycoerythrin or FITC (Vector, 1:1000) were used. Sections were counterstained with DAPI and mounted with Mowiol 488 (Calbiochem). Microscopy was performed on either a Nikon Eclipse (TE200S) inverted fluorescence or a combined confocal/multi-photon (Spectraphysics laser, BioRad MRC1024MP) inverted system. Images were captured in a blinded fashion and quantitative intensity (expression) data was obtained by Metamorph Imaging software (Universal) as previously described (4).

### Morphometric Analysis.

Analysis was performed in a blinded fashion on coded slides as described, using a macro developed by Dr. Rubin M. Tuder (U Colorado) for Metamorph (26, 27).

### Measurement of Lung Volume.

Lung volume was measured using the flexiVent system (Seireq, Montreal, Canada). Mice were anesthetized with inhaled isoflurane in oxygen and orotracheally intubated with a 20 gauge intravenous cannula under direct vision. A good seal was confirmed by stable airway pressure during a sustained inflation. Isoflurane anesthesia was maintained throughout the measurements, and the mice were hyperventilated to eliminate spontaneous ventilation.

### Western Blotting.

Lung tissue was homogenized in RIPA buffer with protease inhibitors on ice and proteins were isolated by centrifugation at 16,000×g for 10 minutes at 4° C. Proteins were loaded in equal amounts (10-30 μg) as determined by BCA protein concentration assay (Pierce. Rockford, Ill.). Total proteins were separated by SDS-PAGE using Criterion gels (Bio-Rad) followed by immunoblotting. Briefly, samples were mixed with Laemmli buffer, heated at 95° C. for 5 min and loaded onto 4-20% SDS-PAGE gels. Proteins were separated by electrophoresis and blotted onto PVDF membranes (Millipore). Non-specific binding was reduced by blocking the membrane in Protein Free Blocking buffer (Pierce) or TBS/0.1% tween-20/5% nonfat dry milk. Primary antibodies were diluted in a sodium phosphate buffer containing 50 mM sodium phosphate, 150 mM NaCl, 0.05% Tween-20, 4% BSA, and 1 mM sodium azide. Primary antibodies and their dilutions are as follows: ERK1/2 (1:2000; Cell Signaling), phospho-ERK1/2 (1:1000; Cell Signaling), p38 (1:1000; Cell Signaling), phospho-p38 (1:1000; Cell Signaling). JNK (1:1000; Cell Signaling), phospho-JNK (1:1000; Cell Signaling), vinculin (1:5000: Calbiochem), or β-actin (1:30,000; Sigma). Blots were washed with TBS+0.1% Tween-20 and incubated with HRP-conjugated secondary antibodies to rabbit (1:10,000; Amersham; Piscataway, N.J.) or mouse (1:10,000; Amersham) in 5% dry milk in TBST. Blots were detected using ECL-plus (Amersham) or SuperSignal (Pierce).

### Hematopoletic Progenitor Cell Analysis.

The absolute numbers and cell cycling status of granulocyte macrophage (CFU-GM), erythroid (BFU-E), and multipotential (CFU-GEMM) progenitor cells was calculated as previously reported (28, 29). In short, BM cells were flushed from femurs of control and treated mice, and nucleated cellularity calculated per femur. Femoral cells were treated in vitro with control medium, or high specific activity tritiated thymidine as a 30 minute pulse exposure, washed, and plated at 5×10⁴ cells/ml in 1% methylcellulose culture medium with 30% fetal bovine serum (FBS, Hyclone, Logan, Utah), and recombinant human erythropoietin (Epo, 1 U/ml, Amgen Corp, Thousand Oaks, Calif.), recombinant murine stem cell factor (SCF, 50 ng/ml, R & D Systems, Minneapolis, Minn.), and 5% vol/vol pokeweed mitogen mouse spleen cell conditional medium (29). Semi-solid cell cultures were placed in culture at 5% CO₂ at lowered (5%) O₂ in a humidified chamber, and CFU-GM-, BFU-E-, and CFU-GEMM-colonies scored after 7 days incubation. The number of colonies and femoral nucleated cellularity was used to calculate numbers of progenitors per femur. The high specific activity tritiated thymidine kill assay allows an estimate of the cell cycling status of progenitors by analysis of the percent progenitors in S-phase at time cells were removed from mice and plated (29).

### Lung Endothelial Cells.

Primary human lung microvascular endothelial cells were obtained from Lonza (Allendale, N.J.) and maintained in culture medium consisting of EMB-2, 5% FBS, 0.4% hydrocortisone, 1.6% hFGF, 1% VEGF, 1% IGF-1, 1% ascorbic acid, 1% hEGF, 1% GA-100, and 1% heparin at 37° C. in 5% CO₂ and 95% air. Experiments were performed up to passage 10 with cells at 80-100% confluence.

### CS Extract Preparation.

An aqueous CS extract was prepared from filtered research grade cigarettes (1R3F) from the Kentucky Tobacco Research and Development Center at the University of Kentucky. A stock (100%) CS extract was prepared by bubbling smoke from 2 cigarettes into 20 ml of basal culture medium (EBM2; Lonza) at a rate of 1 cigarette per minute to 0.5 cm above the filter, using a modified method developed by Carp and Janoff (30). The extract's pH was adjusted to 7.4, followed by filtration (0.2 m, 25 mm Aerodisc; Pall, Ann Arbor, Mich.) and used in cell culture experiments within 20 min. A similar procedure was used to prepare the control extract, replacing the CS with ambient air.

### Endothelial Cell Wound Repair Assays.

Wounding of cultured cells was performed using the Electric Cell Impedance System (ECIS, Applied Biophysics; Troy, N.Y.). Human lung microvascular endothelial cells were grown as detailed above on gold microelectrodes (8W1E) until confluent. Cells were pretreated for 2 hr in basal medium or in conditioned medium collected from cultured adult human ASC (50% v:v). Cells were then treated wounded via a linear electrical injury applied via ECIS, in the presence or absence of CS extract (4%). Wound repair was quantified by measuring cellular resistance over time and normalizing it to the time of wounding, reporting the slope of the TER recovery until monolayer confluence was achieved.

### Statistical Analysis.

Statistics were performed with SigmaStat software using ANOVA with Student-Newman-Keuls post hoc test, or Student's t-test. Statistical difference was accepted at p<0.05.

### 1. ASC Characterization and Localization in the Lungs Following Systemic Delivery.

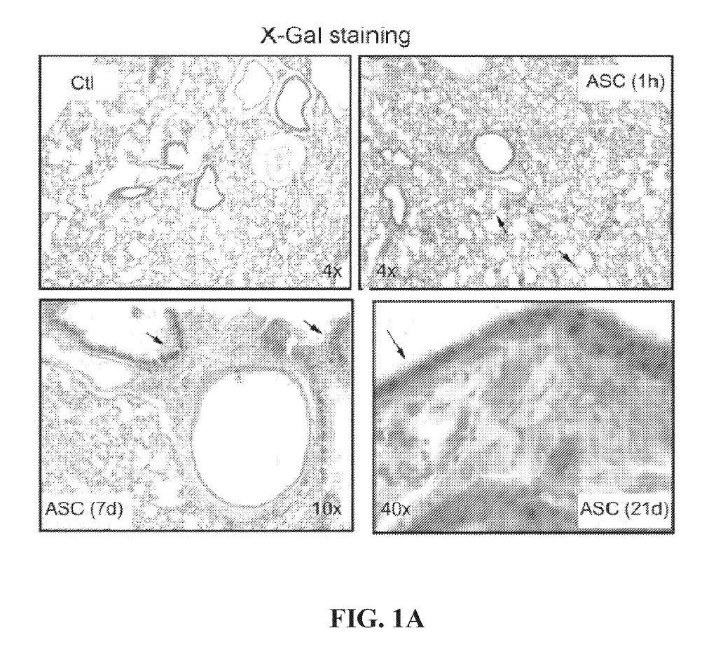

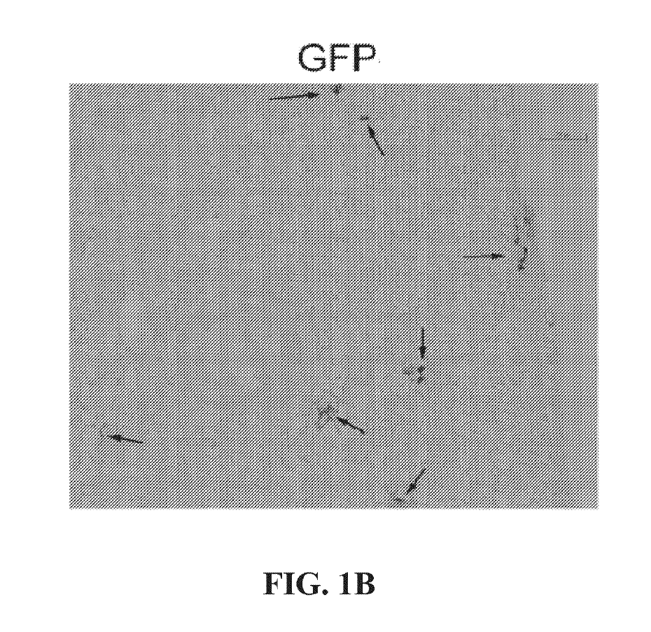

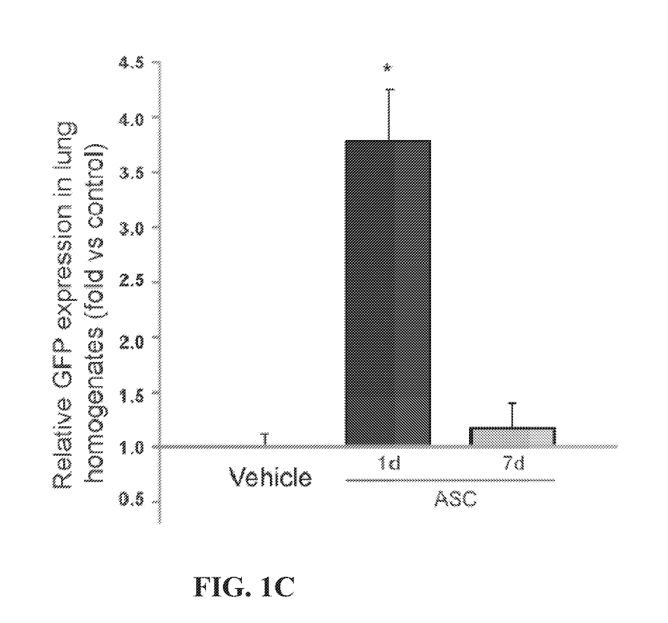

Referring now to FIGS. 1A, LB and IC, briefly, localization of β-galactosidase-expressing murine ASC (dark spots) on lung sections imaged at the indicated magnification following fixation and staining with X-Gal and hematoxyllin. Lungs of Apo E mice were harvested at the indicated time (1 h, 7 d, and 21 d) following 5×10⁵ ASC or control vehicle (Ctl) administration. Note (arrows) the presence of ASC in the lung parenchyma (1 h) and among the bronchial epithelial layer (7 d and 21 d). Localization of GFP-expressing murine ASC (arrow) on lung sections following fixation and immune staining with GFP antibody (B) and counterstaining with hematoxylin (B). Lungs of DBA/2J mice were harvested 7 days following ASC administration (3×10⁵). Note (arrows) the presence of ASC intercalated among the bronchial epithelial layer (B) and in the lung parenchyma (C). Referring now to FIG. 1C, bansize 100 μm, the abundance of DiI-labeled murine ASC detected by flow cytometry of cells obtained from whole lung homogenates following digestion and disintegration. Lungs were harvested 1- and 7-days following ASC administration (3×10⁵) or vehicle in DBA/2J mice previously exposed to CS for 2 weeks. *p<0.05 versus vehicle control; ANOVA.

Initial studies of the distribution of ASC following systemic administration were conducted using ROSA26 mouse-derived ASC expressing β-galactosidase under the control of an unknown endogenous promoter delivered intravenously into non-B-galactosidase expressing mice bearing a homozygous deletion of the ApoE locus. Tissues of these animals were stained for β-galactosidase expression at 1, 7, and 21 days following delivery. Gross inspection 1 h following administration revealed a predominantly pulmonary localization, with a pattern of distribution consistent with intravascular trapping (FIG. 1A), which was confirmed histologically by the presence of ASC in the lung parenchyma. Interestingly, evaluation at 7 and 21 days following ASC delivery demonstrated focal areas of staining consistent with incorporation of B-galactosidase-expressing cells in the airway epithelium, including that of medium and large-sized airways (data not shown).

In separate homing experiments, autologous GFP-labeled mice ASC (3×10⁵ cells) were administered systemically via intravenous injection to DBA/2J mice. Using immunohistochemistry, GFP-labeled cells were detected in the lung alongside resident cells in both large airway epithelial and sub-epithelial structures (FIG. 1B) as well as in parenchyma vascular and alveolar structures at 1 week following their administration (data not shown). For a more quantitative assessment, the homing of Vybrant DiI-labeled ASC to the lung was assessed by flow cytometry of disintegrated lungs at days 1 and 7 following a single injection of ASC (FIG. 1C). Consistent with previous experiences, initial retention of human ASC in the lung following systemic delivery in ApoE mice, the injected DiI-labeled mouse ASC were found in 3-fold higher numbers in the lungs at day 1, compared to 7 days after injection (p<0.05). It is not known whether the persistence of ASC in the lungs is required for their putative regenerative effects in the lung. The effects of repetitive injection of ASC were sufficient to prevent airspace enlargement in CS-induced emphysema, the disease model of highest clinical relevance. To ensure that all expected components of the emphysematous process, including inflammatory elements remained intact in these studies DBA/2J mice with isogenic mouse-derived ASC were used.

2. Treatment with ASC Decreased CS-Induced Lung Inflammation, Apoptosis, and Airspace Enlargement

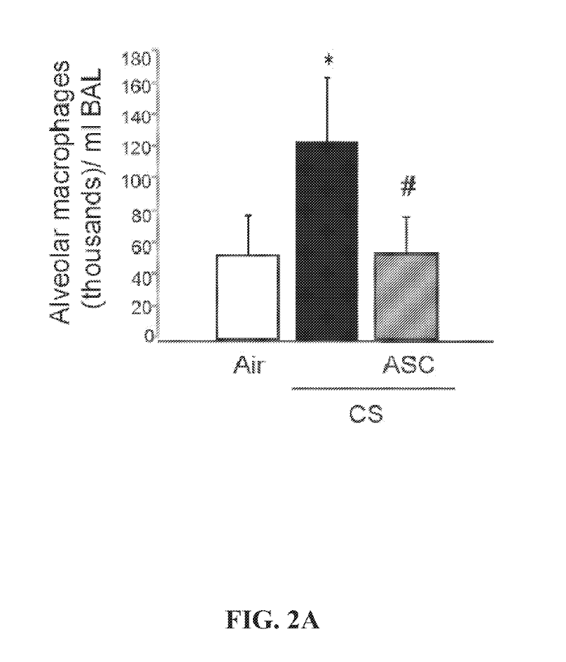

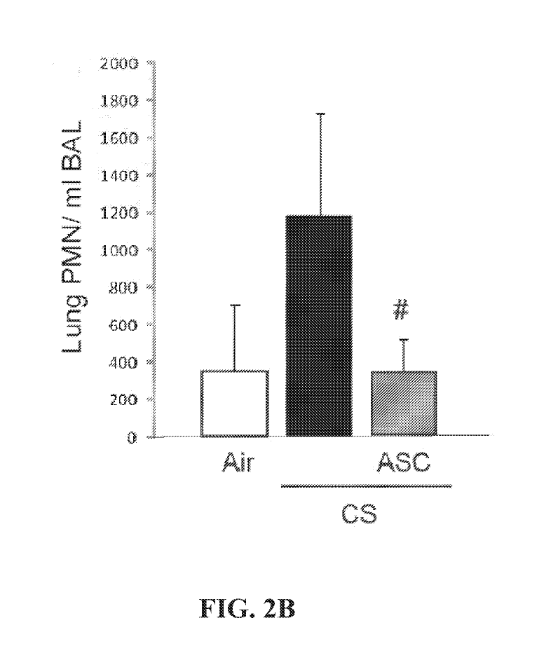

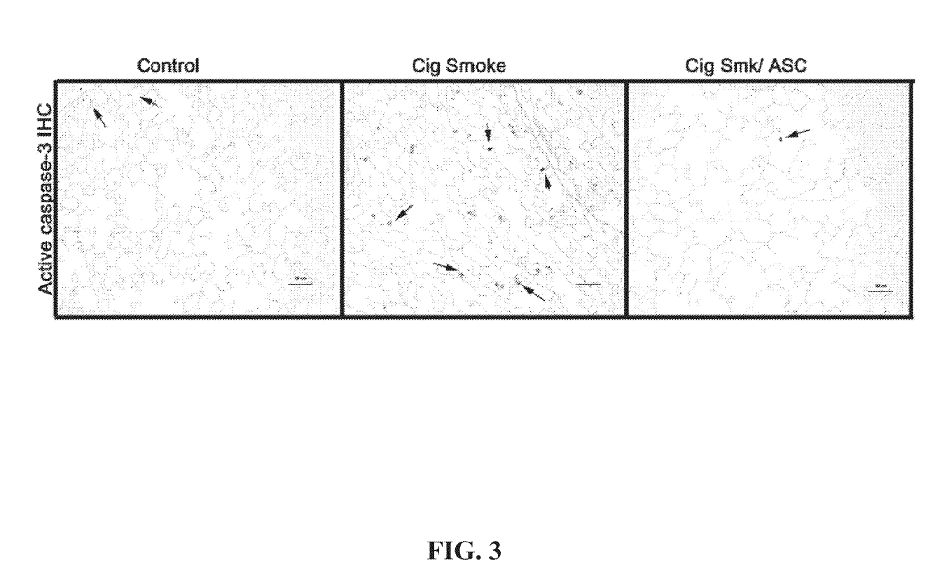

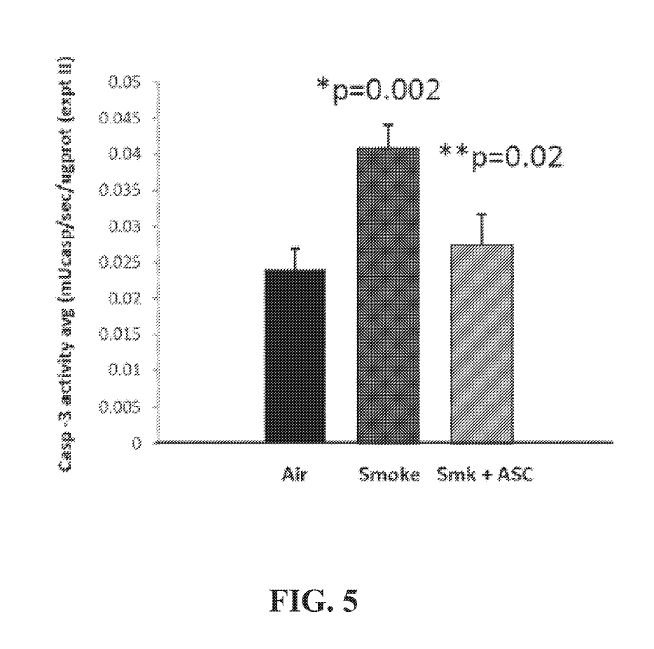

Referring now to FIGS. 2A and 2B, briefly, abundance of inflammatory cells alveolar macrophages (FIG. 2A) and polymorphonuclear cells (FIG. 2B) in the bronchoalveolar lavage (BAL) fluid collected from DBA/2J mice exposed to CS or ambient air (Air) for 4 months (n=8-121 group) and treated with ASCs (3×10⁵ cells infused intravenously every other week, during the month 3 and 4 of CS exposure). *p<0.05 versus control; #p<0.05 versus CS; ANOVA. The abundance of active caspase-3-expressing cells in lung parenchyma was measured (data not shown); by automated image analysis of lung sections immunostained with a specific antibody (FIG. 3). Referring now to FIG. 5, graph of caspase-3 activity measured in hydrasates made from lungs exposed to air, CS and CS+ASC.

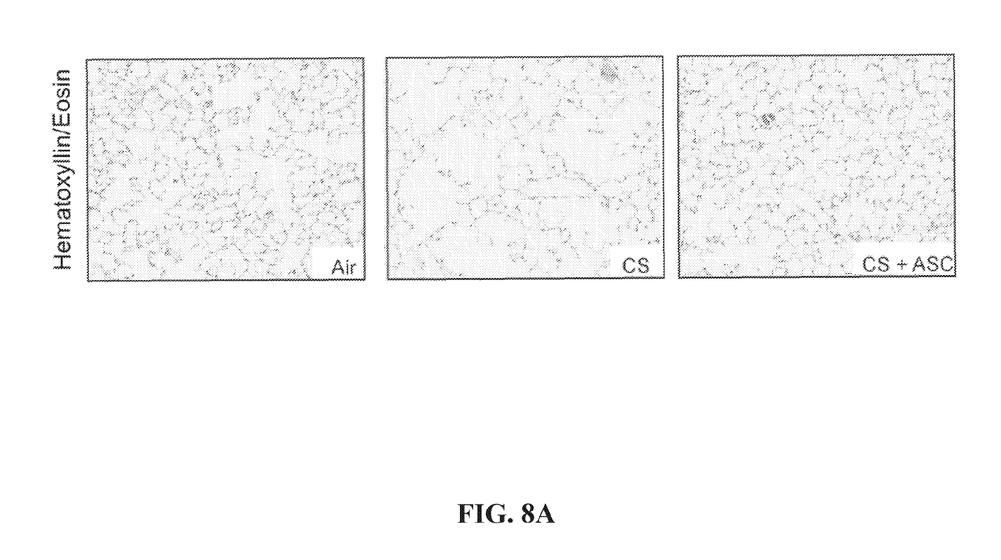

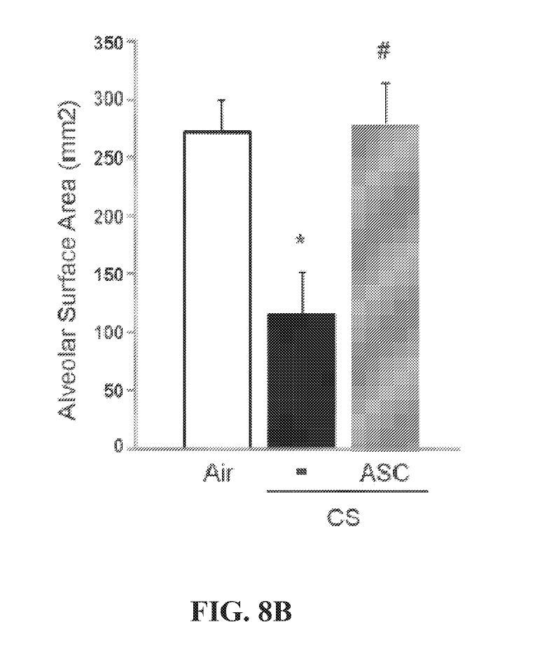

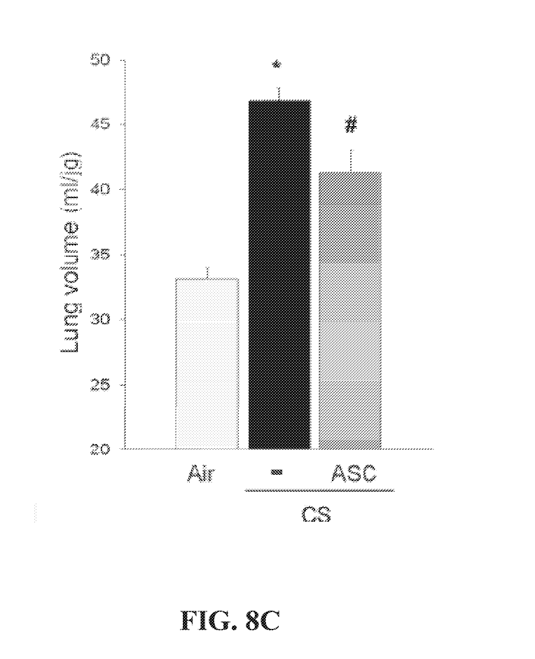

Referring now to FIGS. 8 A, B and C, alveolar airspaces stained with hematoxylin/eosin on fixed lung sections from mice exposed to CS or ambient air for 4 months. DBA/2J mice were treated with ASC (3×10⁵ cells per injection, injected intravenously every other week), during the month 3 and 4 of CS exposure. Note the increased airspaces in the CS-exposed mice and the smaller airspaces in the CS-exposed mice treated with ASC. Referring now to FIG. 8B, alveolar surface area calculated by standardized morphometry of alveolar spaces on coded slides (mean+SEM; *p<0.05 versus air control; #p<0.05 versus CS; ANOVA). Referring now to FIG. 8C, lung volumes measured in anesthetized and intubated DBA/2J mice (n=5-10) at 4 months following CS exposure (mean+SEM; *p<0.05 versus air control; #p<0.05 versus CS; ANOVA).

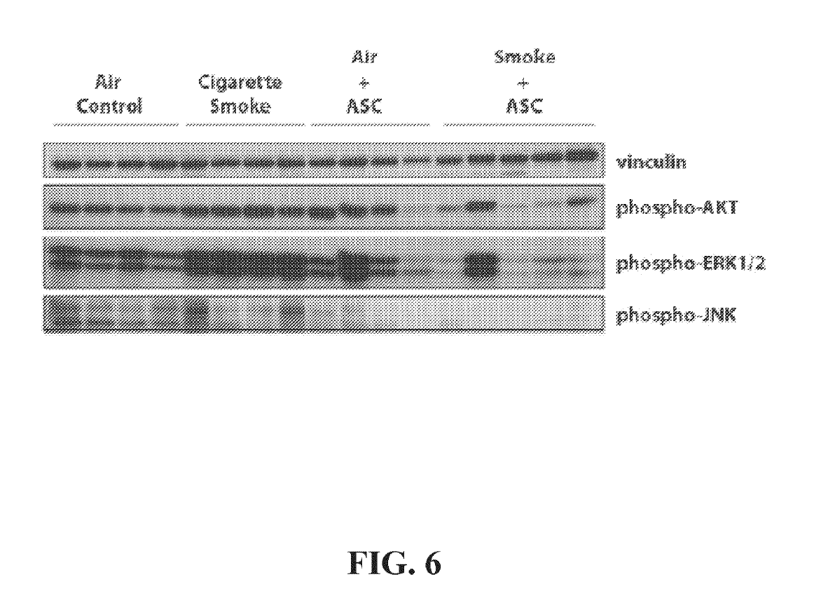

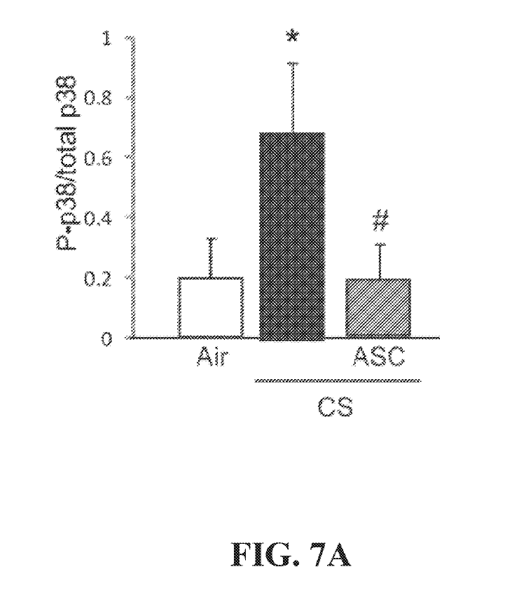

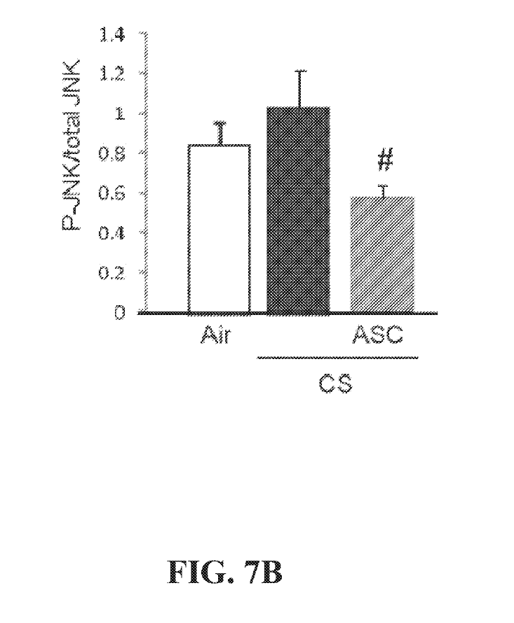

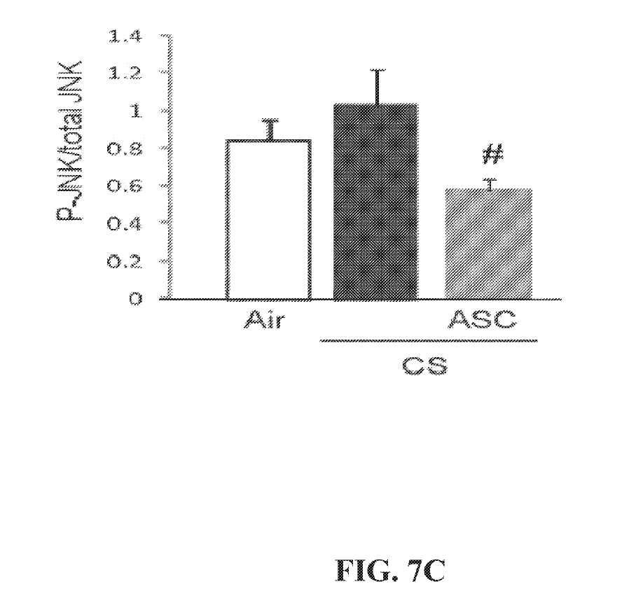

Referring now to FIG. 6 and FIGS. 7A, B and C, briefly, levels of p38 MAPK, JNK1, and Akt activation were measured by densitometry. The amounts of phosphorylated proteins relative to total levels of the respective proteins detected by immunoblotting of total lung homogenates with specific antibodies are reported. The lungs from DBA/2J mice were harvested following 4 months of air or CS exposure. A third group was treated with ASC (3×10⁵ cells per injection, injected intravenously every other week), during the month 3 and 4 of CS exposure (mean+SEM; n=4-6 lung samples from individual mice; *p<0.05 versus air control; #p<0.05 versus CS; ANOVA). Treatment with ASC abrogated the phosphorylation of p38 MAPK and attenuated JNK1 and AKT activities induced by the chronic CS exposure.

DBA/2J mice were exposed to CS or ambient air for 4 months; while a third group of mice, also exposed to CS in parallel, were given ASC collected from littermate mice, expanded ex vivo, and administered by intravenous injection every other week during the last 2 months of the 4 month CS exposure. In a second similar experiment, a fourth group of CS-exposed mice received ASC carrier, as a vehicle control. As expected, CS exposure (4 months) in the DBA/2J mice increased inflammation, measured by an elevated number of inflammatory cells (macrophages and polymorphonuclear cells) in the bronchoalveolar lavage (BAL), (FIGS. 2A and 2B) increased alveolar cell apoptosis, measured by caspase-3 activity and immunohistochemistry (FIG. 3) and caused significant alveolar space enlargement, measured by the standardized automated morphometry of alveolar structure on H/E stained lung section, when compared to control animals exposed to ambient air (FIG. 8A).

In the groups receiving systemic injections of ASC, there was an attenuation of the CS-induced increase in the number of macrophages and PMNs in the BAL (FIGS. 2A and 2B). ASC treatment attenuated the enzymatic activity of caspase-3 in total lung homogenates by more than 30% (p=0.02), and markedly decreased the CS-induced active caspase-3 expression in the lung parenchyma, measured by immunohistochemistry when compared to the mice who did not received ASC or only received vehicle control (FIG. 3). These protective effects were associated with a significant decrease in alveolar space size compared to the group exposed to CS alone (FIG. 8A), which was reflected by a significant decrease in the mean linear intercepts (MLI) from to 40.5±1 μm to 36.3±0.7 μm (p=0.01), a significant increase in alveolar surface area from 115.7±36 mm² to 280.1±34 mm² (p=0.004) (FIG. 8B), and a significant attenuation of lung volume enlargement (p=0.01) (FIG. 8C). The protective effects of ASC on lung inflammation, apoptosis, and alveolar integrity were associated with biochemical evidence of modulation of the CS-induced p-38 MAPK (FIG. 7A) and attenuated JNK1 (FIG. 7B) and AKT (FIG. 7C) activity induced by chronic exposure to CS.

The result was significant alveolar space enlargement, measured by the standardized automated morphometry of alveolar structures on H/E-stained lung sections, when compared to control animals exposed to ambient air (FIG. 8A). Referring again to FIGS. 2A and 2B, in the group receiving systemic injections of ASC, there was an attenuation of the CS-induced increase in the number of macrophages and PMNs in the BAL. ASC treatment attenuated the enzymatic activity of caspase-3 in total lung homogenates by more than 30% (p=0.02) (data not shown), and markedly decreased the CS-induced active caspase-3 expression in the lung parenchyma, measured by immunohistochemistry (FIG. 3) and reduced caspase-3 activity (FIG. 5) when compared to the CS-exposed mice who did not receive ASC or who only received vehicle control.

3. Treatment with ASC Prevents CS-Induced Weight Loss in Mice

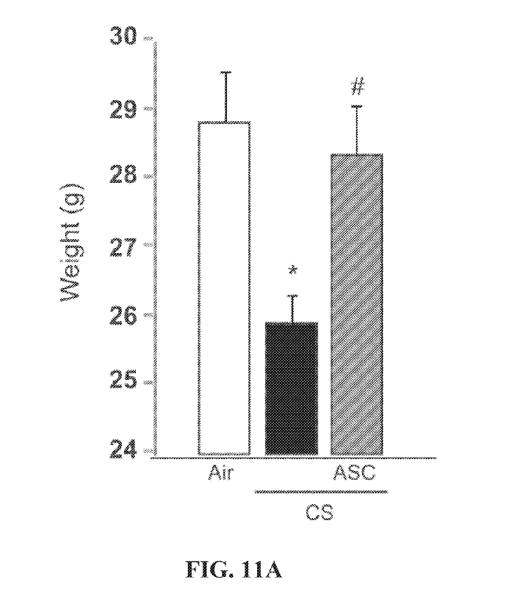

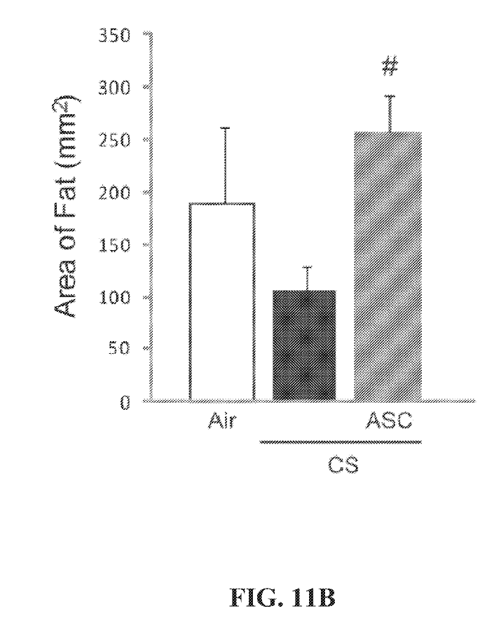

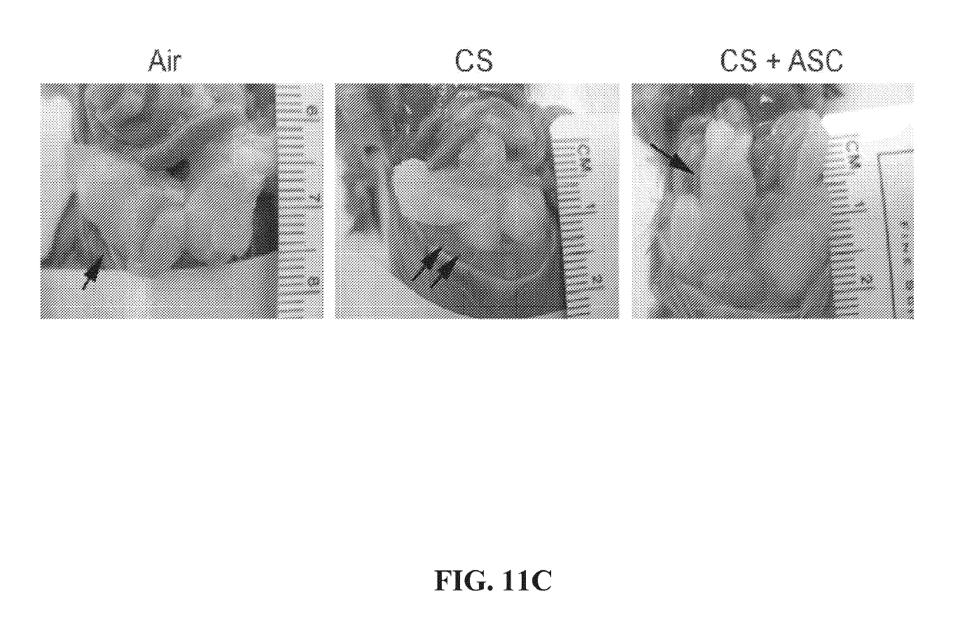

Referring now to FIGS. 11 A, B and C. The body weights of DBA/2J mice following 4 months of air or CS exposure were measured. A third group of mice were treated with ASC (3×10⁵ cells per injection, injected intravenously every other week), during the month 3 and 4 of CS exposure (mean+SEM; n=10-12 *p<0.05 versus air control; #p<0.05 versus CS; ANOVA) (FIG. 11A). Referring now to FIG. 11B, the abundance of abdominal fat (A; mean+SEM; n=3-6 *p<0.05 versus air control; #p<0.05 versus CS; ANOVA), was measured at 4 months. Referring now to FIG. 11C mice were dissected and photographed to determine the distribution of fat within the animals' bodies. A decrease in the amount of abdominal fat in the CS-exposed mice (double arrows), compared to control mice and to ASC-treated CS-exposed mice (arrows) was clearly noted, there did not appear to be a notable difference in the amount of fat or its distribution between animals in the control and those treated with ASC.

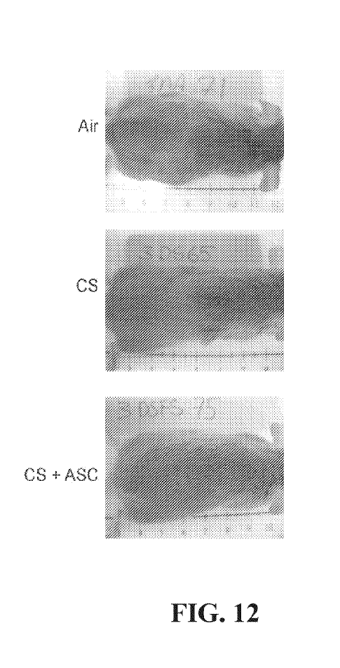

Referring now to FIG. 12, mice were photographed following 4 months of exposure to air or to CS exposure a group of mice exposed to CS were treated with ASC during the last 2 months of exposure. Note the smaller size (girth) of CS-exposed mice and the similar size of ASC-treated CS-exposed mice compared to control mice.

As previously noted (20), chronic CS exposure caused a significant decrease in body weight, reaching 10% after 4 months of exposure (p=0.003) compared to mice of similar age and sex exposed to ambient air for the same duration of time (FIG. 11A). Interestingly, CS-exposed mice treated with ASC during the last 2 months of exposure had no significant weight loss compared to ambient-air exposed control animals (FIG. 11A and FIG. 12). Referring now to FIGS. 11B and 11C, when examined macroscopically, the area of fat measured from coded (blinded) photographs of abdominal subcutaneous region, the ASC-treated mice had a significant increase (p<0.05) in the abundance of subcutaneous fat compared to the untreated CS-exposed mice. Macroscopically, no difference in the body distribution of fat was noted compared to that in control mice (FIG. 12).

4. Treatment with ASC Restored the Bone Marrow (BM) Dysfunction Induced by CS in Adult Mice.

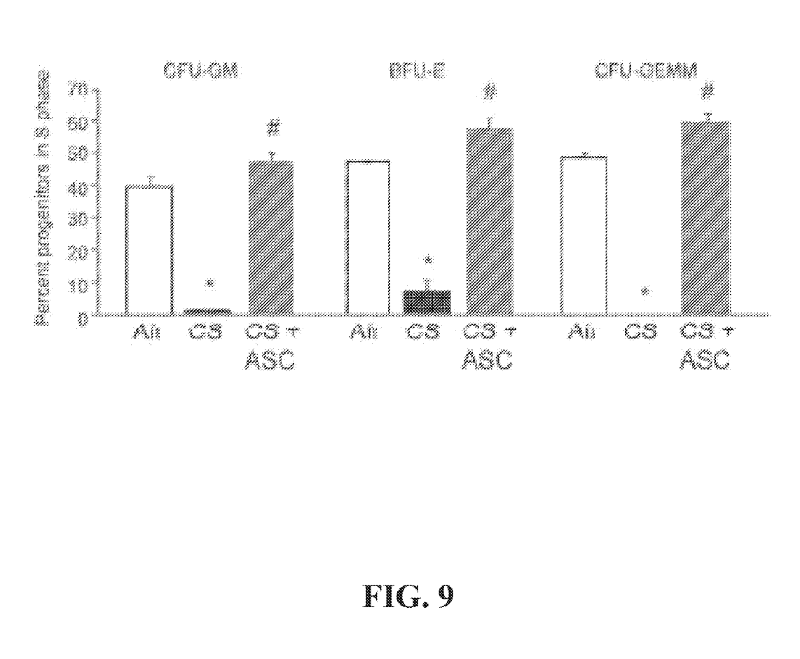

Referring now to FIG. 9, The absolute numbers of nucleated cells, and the following hematopoietic progenitor cells: colony forming unit-granulocyte, monocyte, CFU-GM, burst-forming unit-erythroid, BFU-E, colony forming unit-granulocyte, erythrocyte, monocyte, and megakaryocyte, CFU-GEMM, and cycling status (=percent cells in S-phase) of these progenitors in DBA/2J mice following 4 months of air or CS exposure, with a third group treated with ASC (3×10⁵ cells per injection, injected intravenously every other week), during the month 3 and 4 of CS exposure (mean+SEM; n=4-6; *p<0.05 versus air control; #p<0.005 versus CS; ANOVA) were determined.

One of the less widely appreciated and studied systemic affects of CS exposure is that exposure to CS suppresses BM function (31, 32). To evaluate the capability of ASC to modulate the toxic effects of chronic CS exposure on hematopoiesis, BM was harvested from the femora of DBA/2J mice exposed to CS for 4 months. The mice exposed to CS were divided into 2 groups one group received only the carrier (control) while the other group was treated with ASC in its carrier.

Referring to FIG. 9, CS exposure resulted in a marked and significant reduction in absolute numbers of bone marrow CFU-GM, BFU-E and CFU-GEMM cells. The cells exposed to CS without treatment with ASC, were also in a slow or non-cycling state. In stark contrast cells from animals that were never exposed to CS and cells from animals treated with ASC during the last 2 months of CS exposure more BM progenitor cells and these cells were much more likely to be in S-phase. These results demonstrate that the effects of CS on bone marrow progenitor cell populations can be fully or nearly completely counteracted by treatment with ASC.

5. Treatment with Human ASC Decreased VEGFR-Inhibitor Induced Airspace Enlargement in Immunodeficient Mice.

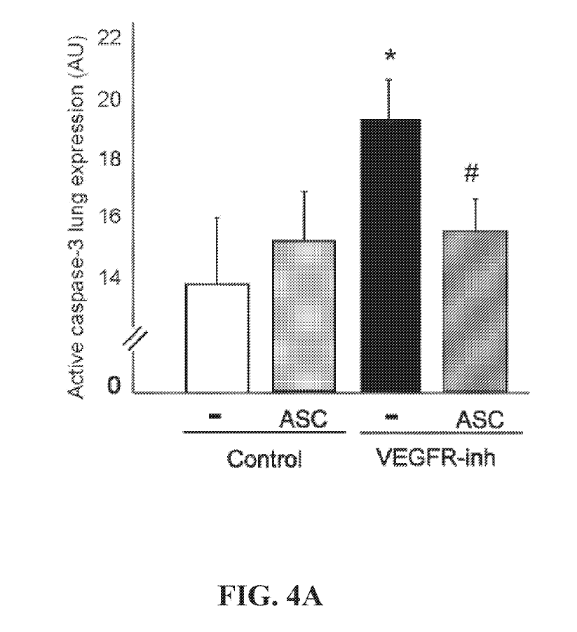

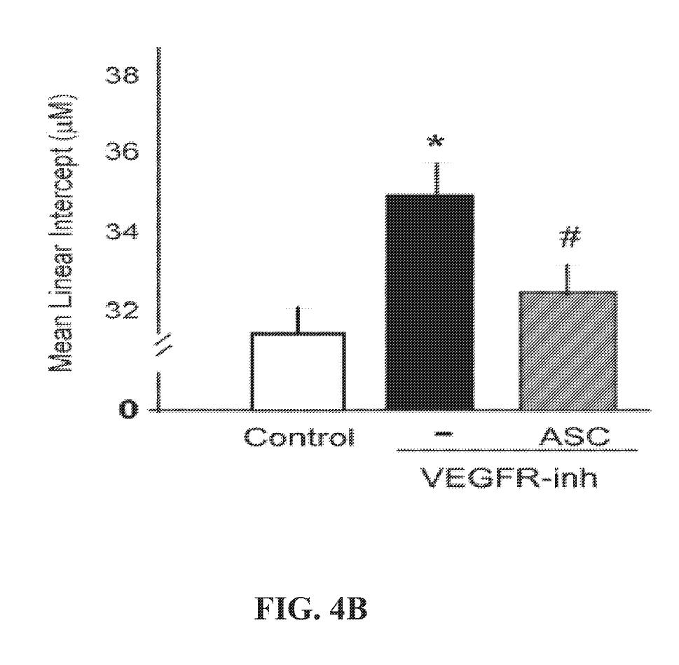

Referring now to FIGS. 4A and 4B, briefly, lung apoptosis was quantified by abundance of active caspase-3-expressing cells in lung parenchyma (at 4 weeks) in animals (Nod-SCID NS2 mice) who received a single dose of VEGF receptor inhibitor (SU5416, 20 mg/kg; sq) or its vehicle control (CMC), and who were treated with human adult ASC (3×10⁵, intravenous injection) on day 3 following VEGFR inhibition; (A; mean arbitrary units (AU)+SEM; *p<0.05 versus vehicle (control); #p<0.05 versus ASC-untreated (−) animals who received the VEGFR-inhibitor; ANOVA). Quantification was achieved by automated image analysis of coded lung sections immunostained with a specific active caspase-3 antibody (image not shown). Mean linear intercepts calculated by standardized morphometry of alveolar spaces on coded slides of alveolar airspaces stained with hematoxylin/eosin on fixed lung sections from mice exposed to CMC vehicle or VEGFR-inhibitor air for 24 weeks and treated with ASC as previously described. (mean+SEM; *p<0.05 versus CMC control; #p<0.05 versus VEGFR-inhibited mice; ANOVA).

The mechanism(s) by which ASC exerted their protective local and systemic effects in the CS model may include paracrine release of survival and growth factors, including VEGF (33, 34), which oppose the excessive apoptosis noted in response to CS exposure. This hypothesis, was tested using a complementary model of emphysema driven by apoptosis due to decreased VEGF availability. It was previously demonstrated that VEGFR blockade with SU5416 (20 mg/kg; subcutaneously) caused significant increases in airspace enlargement in C57Bl/6 mice that peaked at 28 days (3). This airspace enlargement is dependent on alveolar cell apoptosis (1, 3), detected not only in endothelial but also in epithelial cell types (3), making this model ideally suited to address whether ASC treatment is sufficient to overcome a VEGF-deprived state and influence endothelial survival. In addition, to investigate whether not only the mouse, but also the human adult ASC are efficient at protecting against lung apoptosis, we employed immunodeficient Nod-SCID interleukin 2 receptor gamma chain-deficient (NS2) mice. Pilot experiments using this mouse demonstrated that the immunotolerant NS2 mouse is susceptible to development of airspace enlargement as a result of VEGFR blockade. Indeed, administration of SU5416 (20 mg/kg, subcutaneously) showed the NS2 mice exhibited a significant increase in alveolar enlargement at 21 days compared to vehicle (carboxymethylcellulose (CMC) controls in both male and female adult mice (data not shown).

Since systemically delivered ASC preferentially lodge and engraft in the lungs of mice 24 h following systemic delivery, human ASC (3×10⁵ cells we administered; intravenous injection) at day 3 following VEGFR inhibition in adult NS2 female mice, a time at which lung apoptosis is increasing in this model, peaking between 3-7 days of VEGFR administration (3). GFP-labeled human ASC were detected in the lungs of NS2 mice 3 days after injection (day 6 of VEGFR blockade), as determined by GFP immunoblotting of total lung homogenates (data not shown).

Referring now to FIGS. 4A and 4B, at 28 days, the VEGFR blockade-induced increase in apoptosis, measured by image analysis and quantification of the immunohistochemical expression of active caspase-3 in the lung parenchyma was significantly attenuated by 75% (p=0.03) following treatment with a single injection of human adult ASC. Furthermore, the VEGFR-blockade-induced alveolar enlargement was significantly decreased, measured by a 70% improvement (p=0.006) in mean linear intercepts following the systemic administration of human adult ASC (data not shown). These results suggested ASC have prominent protective anti-apoptotic effects in the lung, thus, overcoming the specific effects of VEGF inhibition.

### 6. Human ASC-Conditioned Medium Improved the Repair of Lung Endothelial Cells Monolayers In Vitro.

To further characterize a potential paracrine protective effect of ASC towards injured lung microvascular endothelial cells, adult human ASC-conditioned medium (ASC-CM) in an in vitro model of lung endothelial injury was tested. The integrity of the normally tight cultured lung endothelial cell monolayers can be tracked in real time by measuring the trans-endothelial electrical resistance (TER) of cells grown on microelectrodes, utilizing the electrical cell impedance system (ECIS). Utilizing this approach, the effect of ASC-CM on lung endothelial cell wound repair following wounding induced by a linear electrical injury applied through microelectrodes in contact with the monolayer was determined. Following wounding, which is characterized by a sudden decrease in TER, the monolayer repairs via both cell growth and migration of endothelial cells from the wound edges towards the “wound” (35), which is reflected by a gradual restoration of TER towards that of confluent monolayers. Cell monolayers grown at confluence were “wounded” via a linear electrical injury applied through microelectrodes in contact with the monolayer.

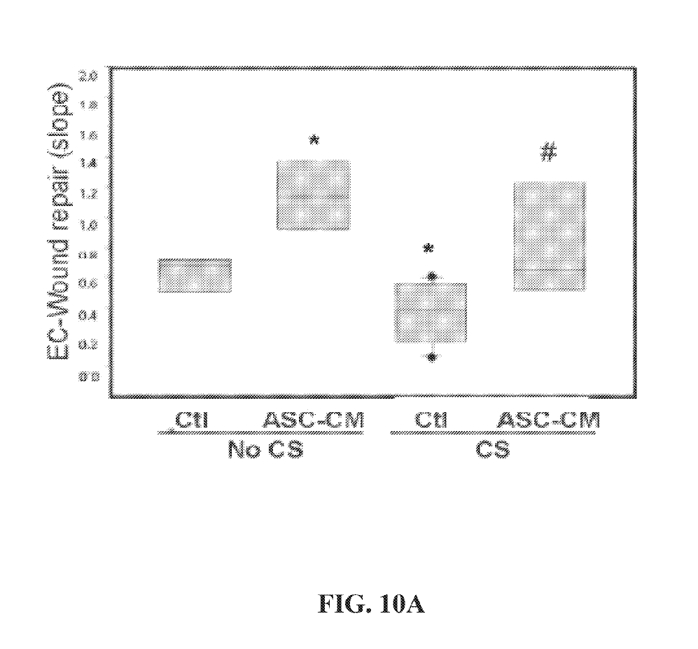

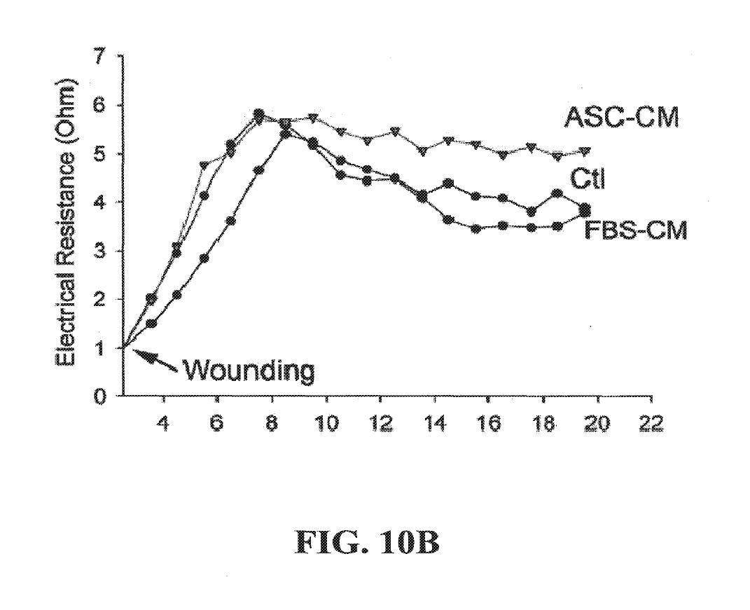

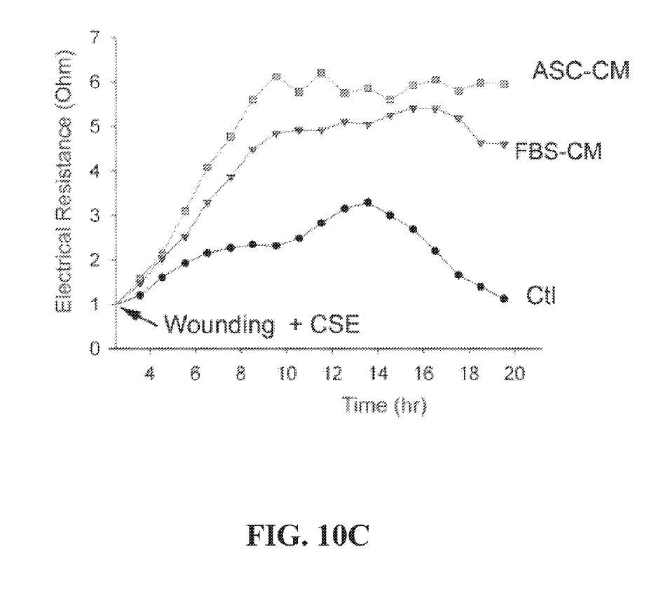

Referring now to FIGS. 10 A, B and C. Briefly, wound injury repair measured by the recovery of trans-cellular electrical resistance (TER) across a confluent monolayer of primary human lung microvascular endothelial cells grown on gold microelectrodes using the Electric Cell-Substrate Impedance Sensing (ECIS) system. A linear electrical injury was applied at time 0 (B-C, arrow) and the slope of TER recovery to plateau was compared for cells maintained in their regular growth medium, or in medium supplemented with conditioned medium from ASC cells (ASC-CM; 50%), in the absence and presence of CS extract (4%); A; boxplot with medians; n=4 independent experiments; p<0.01 2-Way ANOVA for the effect of CS and ASC-CM; *p<0.005 versus untreated wounded control cells; #p<0.005 versus untreated wounded CS-exposed cells. FIGS. 10 B and C. Kinetics of normalized TER (to the TER at time of wound application) (mean; n=3-4 independent experiments) in unexposed cells (B) or in cells exposed to CS (FIG. 10 C) wounded at time 0 (arrow), which were untreated, grown in their control medium (Ctl; black line) or treated with ASC-CM (green line) or with control serum-containing media (FBS-CM, 20%; red line). Note the effect of CS extract on both the slope and the attained plateau levels of TER recovery in wounded lung endothelial cells and the protective effects of both ASC-CM and serum on the slope of TER recovery, with ASC-CM-specific effects on the plateau TER.

Referring now to FIGS. 10A and 10B, pretreatment of primary human lung microvascular endothelial cell monolayers with ASC-CM significantly (p=0.003) enhanced the TER recovery following wounding compared to untreated cells. Referring now to FIGS. 10A and 10C, interestingly, in the presence of a CS extract, which contain the water soluble fraction of CS that mimics its circulating components, there was a marked delay in lung endothelial cell wound healing. Both the slope of TER recovery and the absolute TER attained at full recovery following wounding were significantly blunted compared with wounded endothelial cells exposed to ambient air-extract control. Strikingly, endothelial cell monolayers repaired the wound significantly faster in the presence of ASC-CM, even during concomitant CS extract exposure (FIG. 10A). Since the ASC-CM includes serum necessary for their growth, and since serum itself has numerous growth factors, the effect of the control conditioned medium which contained serum on wound repair was investigated.

Referring now to FIGS. 10B and 10C, although serum exerted a marked protective effect on the slope of wound repair, only cells treated with ASC-CM sustained their monolayer barrier function attained following wounding. These data suggest that factors secreted by adult human ASC exert protective effects against lung endothelial cell damage and may antagonize the injurious effects of CS exposure.

### 7. Cigarette Smoke Exposure

Mice susceptible to cigarette smoke-induced emphysema were exposed to cigarette smoke for various periods of time, from 1 day to 4 months. Cigarette smoke exposure for 4 weeks increased caspase-3 activity and the content of ceramide in lungs, and thus increased apoptotic activity in DBA2 mice, long preceding the increases in airspaces typical of emphysema that occurred at 4 months of cigarette smoke exposure in this strain. Adult adipose stem cells which were obtained from littermate DBA2 mouse adipose tissue and were subsequently maintained in culture conditions and subsequently counted and given as treatment to mice which were exposed to cigarette smoke. Administration by intravenous injection of adult adipose stem cells every other week resulted in inhibition of airspace enlargement in mice even when the treatment started after 2 months of cigarette smoking. The mice which were injected adult adipose stem cells had less apoptosis in the lung and less inflammation in the bronchoalveolar lavage induced by cigarette smoking than mice which were not treated. Application of molecular substances directly derived from adult adipose stem cells which were obtained by growing these cells in culture resulted in increased primary human lung endothelial cell growth despite the application of cigarette smoke extract, which inhibited this growth. It is conceivable that adult adipose stem cells or molecular substances directly derived from these cells will help lung endothelial cells withstand the toxic effects of smoking and even repair the damage induced by such exposure.

While the novel technology has been illustrated and described in detail in the figures and foregoing description, the same is to be considered as illustrative and not restrictive in character, it being understood that only the preferred embodiments have been shown and described and that all changes and modifications that come within the spirit of the novel technology are desired to be protected. As well, while the novel technology was illustrated using specific examples, theoretical arguments, accounts, and illustrations, these illustrations and the accompanying discussion should by no means be interpreted as limiting the technology. All patents, patent applications, and references to texts, scientific treatises, publications, and the like referenced in this application are incorporated herein by reference in their entirety.

### REFERENCES

1. Kasahara Y, Tuder R M, Taraseviciene-Stewart L, Le Cras T D, Abman S, Hirth P K, Waltenberger J, Voelkel N F. Inhibition of vegf receptors causes lung cell apoptosis and emphysema. J Clin Invest 2000, 106:1311-1319.
2. Diab K J, Adamowicz J J, Kamoeki K, Rush N I, Garrison J, Gu Y, Schweitzer K S, Skobeleva A, Rajashekhar G, Hubbard W C, et al. Stimulation of sphingosine 1 phosphate signaling as an alveolar cell survival strategy in emphysema. Am J Respir Crit Care Med 2009.
3. Petrache I, Natarajan V, Zhen L, Medler T R, Richter A T, Cho C. Hubbard W C, Berdyshev E V, Tuder R M. Ceramide upregulation causes pulmonary cell apoptosis and emphysema-like disease in mice. Nat Med 2005; 11:491-498.
4. Giordano R J, Lahdenranta J, Zhen L, Chukwueke U, Petrache I, Langley R R, Fidler I J, Pasqualini R, Tuder R M, Arap W. Targeted induction of lung endothelial cell apoptosis causes emphysema-like changes in the mouse. J Biol Chem 2008; 283:29447-29460.
5. Cai L, Johnstone B H, Cook T G, Liang Z, Traktuev D, Cornetta K, Ingram D A, Rosen E D, March K L. Suppression of hepatocyte growth factor production impairs the ability of adipose-derived stem cells to promote ischemic tissue revascularization. Stem Cells 2007; 25:3234-3243.
6. Wei X, Du Z, Zhao L, Feng D. Wei G. He Y, Tan J, Lee W H, Hampel H, Dodel R, et al. Ifats collection: The conditioned media of adipose stromal cells protect against hypoxia-ischemia-induced brain damage in neonatal rats. Stem Cells 2009; 27:478-488.
7. Ishizawa K, Kubo H. Yamada M, Kobayashi S, Numasaki M, Ueda S. Suzuki T, Sasaki H. Bone marrow-derived cells contribute to lung regeneration after elastase-induced pulmonary emphysema. FEBS Lett 2004; 556:249-252.
8. Shigemura N. Okumura M, Mizuno S, Imanishi Y, Nakamura T, Sawa Y. Autologous transplantation of adipose tissue-derived stromal cells ameliorates pulmonary emphysema. Am J Transplant 2006; 6:2592-26(00).
9. Zuk P A, Zhu M. Mizuno H, Huang J, Futrell J W, Katz A J, Benhaim P, Lorenz H P, Hedrick M H. Multilineage cells from human adipose tissue; Implications for cell-based therapies. Tissue Eng 2001; 7:211-228.
10. Rehman J, Li J, Parvathaneni L, Karlsson G, Panchal V R, Temm C J, Mahenthiran J, March K L. Exercise acutely increases circulating endothelial progenitor cells and monocyte-/macrophage-derived angiogenic cells. J Am Coll Cardiol 2004; 43:2314-2318.
11. Rajashekhar G, Traktuev D O, Roell W C, Johnstone B H, Merfeld-Clauss S, Van Natta B, Rosen E D, March K L, Clauss M. Ifats collection; Adipose stromal cell differentiation is reduced by endothelial cell contact and paracrine communication: Role of canonical wnt signaling. Stem Cells 2008; 26:2674-2681.
12. Cai I., Johnstone B H, Cook T G, Tan J, Fishbein M C, Chen P S, March K L. Ifats collection: Human adipose tissue-derived stem cells induce angiogenesis and nerve sprouting following myocardial infarction, in conjunction with potent preservation of cardiac function. Stem Cells 2009; 27:230-237.
13. Rodriguez A M, Pisani D, Dechesne C A, Ture-Carel C, Kurzenne J Y, Wdziekonski B, Villageois A, Bagnis C, Breittmayer J P, Groux H, et al. Transplantation of a multipotent cell population from human adipose tissue induces dystrophin expression in the immunocompetent mdx mouse. J Exp Med 2005; 201:1397-1405.
14. Wei X, Zhao L, Zhong J, Gu H, Feng D, Johnstone B H, March K L, Farlow M R. Du Y. Adipose stromal cells-secreted neuroprotective media against neuronal apoptosis. Neurosci Lett 2009; 462:76-79.
15. Traktuev D O, Prater D N. Merfeld-Clauss S, Sanjeevaiah A R. Saadatzadeh M R, Murphy M, Johnstone B H, Ingram D A, March K L. Robust functional vascular network formation in vivo by cooperation of adipose progenitor and endothelial cells. Circ Res 2009; 104:1410-1420.
16. Guerassimov A, Hoshino Y, Takubo Y. Turcotte A. Yamamoto M, Ghezzo H, Triantafillopoulos A, Whittaker K, Hoidal J R, Cosio M G. The development of emphysema in cigarette smoke-exposed mice is strain dependent. Am J Respir Crit Care Med 2004; 170:974-980.
17. Bartalesi B, Cavarra E, Fineschi S, Lucattelli M, Lunghi B, Martorana P A, Lungarella G. Different lung responses to cigarette smoke in two strains of mice sensitive to oxidants. Eur Respir J 2005; 25:15-22.
18. Agusti A G, Noguera A, Sauleda J, Sala E, Pons J, Busquets X. Systemic effects of chronic obstructive pulmonary disease. Eur Respir J 2003; 21:347-360.
19. Oudijk E J, Lammers J W, Koenderman L. Systemic inflammation in chronic obstructive pulmonary disease. Eur Respir J Suppl 2003; 46:5s-13s.
20. Gosker H R Langen R C, Bracke K R, Joos G F, Brusselle G G, Steele C, Ward K A, Wouters E F, Schols A M. Extrapulmonary manifestations of chronic obstructive pulmonary disease in a mouse model of chronic cigarette smoke exposure. Am J Respir Cell Mol Biol 2009; 40:710-716.
21. Rabkin S, Boyko, E., Streja, D. Relationship of weight loss and cigarette smoking to changes in high-density lipoprotein cholesterol. The American Journal of Clinical Nutrition 1981; 34:1764-1768.
22. Serobyan N, Orlovskayu I. Kozlov V, Khaldoyanidi S K. Exposure to nicotine during gestation interferes with the colonization of fetal bone marrow by hematopoietic stem/progenitor cells. Stem Cells Dev 2005; 14:81-91.
23. Serobyan N, Schraufstatter I U. Strongin A, Khaldoyanidi S K. Nicotinic acetylcholine receptor-mediated stimulation of endothelial cells results in the arrest of haematopoietic progenitor cells on endothelium. Br J Haematol 2005; 129:257-265.
24. Traktuev D O, Merfeld-Clauss S, Li J, Kolonin M, Arap W, Pasqualini R, Johnstone B H, March K L. A population of multipotent cd34-positive adipose stromal cells share pericyte and mesenchymal surface markers, reside in a periendothelial location, and stabilize endothelial networks. Circ Res 2008; 102:77-85.
25. Soriano P. Generalized lacz expression with the rosa26 are reporter strain. Nat Genet 1999; 21:70-71.
26. Tuder R M, Zhen L, Cho C Y, Taraseviciene-Stewart L, Kasahara Y, Salvemini D, Voelkel N F, Flores S C. Oxidative stress and apoptosis interact and cause emphysema due to vascular endothelial growth factor receptor blockade. Am J Respir Cell Mol Biol 2003:29:88-97.
27. Aherne W A, Dunnill M S. Morphometry. London: E. Arnold; 1982.
28. Broxmeyer H E, Orschell C M, Clapp D W, Hangoc G, Cooper S, Plett P A, Liles W C, Li X, Graham-Evans B, Campbell T B, et al. Rapid mobilization of murine and human hematopoietic stem and progenitor cells with amd3100, a cxcr4 antagonist. J Exp Med 2005:201:1307-1318.
29. Cooper S, and Broxmeyer, H. E. Measurement of interleukin-3 and other hematopoietic growth factors, such as gm-csf, g-csf, m-csf, erythropoietin and the potent co-stimulating cytokines steel factor and fit-3 ligand. New York). John Wiley & Sons. Inc.; 1996.
30. Carp H. Janoff A. Inactivation of bronchial mucous proteinase inhibitor by cigarette smoke and phagocyte-derived oxidants. Exp Lung Res 1980; 1:225-237.
31. Liu X, Kohyama T. Kobayashi T, Abe S, Kim H. Reed E C, Rennard S I. Cigarette smoke extract inhibits chemotaxis and collagen gel contraction mediated by human bone marrow osteoprogenitor cells and osteoblast-like cells. Osteoporos Int 2003; 14:235-242.
32. Pandit T S, Sikora L. Muralidhar G, Rao S P. Sriramarao P. Sustained exposure to nicotine leads to extramedullary hematopoiesis in the spleen. Stem Cells 2006; 24:2373-2381.
33. Lee E Y, Xia, Y., Kim. W., Kim, M., Kim, T., Kim, K., Park, B., Sung, J. Hypoxia-enhanced wound-healing function of adipose-derived stem cells: Increase in stem cell proliferation and up-regulation of vegf and bfgf. Wound Repair and Regeneration 2009; 17:540-547.
34. Wang J H, Liu N, Du H W, Weng J S, Chen R H, Xiao Y C, Zhang Y X. [effects of adipose-derived stem cell transplantation on the angiogenesis and the expression of bfgf and vegf in the brain post focal cerebral ischemia in rats.]. Xi Bao Yu Fen Zi Mian Yi Xue Za Zhi 2008:24:958-961.
35. Moy A B, Van Engelenhoven J, Bodmer J, Kamath J, Keese C, Giaever I. Shasby S, Shasby D M. Histamine and thrombin modulate endothelial focal adhesion through centripetal and centrifugal forces. J Clin Invest 1996; 97:1020-1027.
36. Rangasamy T. Cho C Y, Thimmulappa R K, Zhen L, Srisuma S S, Kensler T W, Yamamoto M, Petrache I, Tuder R M, Biswal S. Genetic ablation of nrf2 enhances susceptibility to cigarette smoke-induced emphysema in mice. J Clin Invest 2004; 114:1248-1259.
37. Kang M J. Lee C G, Cho S J, Homer R J, Elias J A. Ifn-gamma-dependent DNA injury and/or apoptosis are critical in cigarette smoke-induced murine emphysema. Proc Am Thorac Soc 2006; 3:517-518.
38. Hoffman A M, Shifren A, Mazan M R, Gruntman A M, Lascola K M, Nolen-Walston R, Kim C F. Tsai L, Pierce R A, Mecham R P, et al. Matrix modulation of compensatory lung regrowth and progenitor cell proliferation in mice. Am J Physiol Lung Cell Mol Physiol 2009.
39. Hegab A E, Kubo H, Yamaya M, Asada M, He M, Fujino N, Mizuno S, Nakamura T. Intranasal hgf administration ameliorates the physiologic and morphologic changes in lung emphysema. Mol Ther 2008; 16:1417-1426.
40. Shigemura N, Okumura M, Mizuno S, Imanishi Y, Matsuyama A, Shiono H, Nakamura T, Sawa Y. Lung tissue engineering technique with adipose stromal cells improves surgical outcome for pulmonary emphysema. Am J Respir Crit Care Med 2006:174:1199-1205.
41. Zhao Y. Ohkawara, H., Rehman, J., Wary, K., Vogel, S., Minshall. R., Zhao, Y., Malik. A. Bone marrow progenitor cells induce endothelial adherens junction integrity by sphingosine-1-phosphate-mediated rac1 and cdc42 signaling. Circ Res 2009; 105:696-704.
42. Di Francia M, Barbier D, Mege J L. Orehek J. Tumor necrosis factor-alpha levels and weight loss in chronic obstructive pulmonary disease. Am J Respir Crit Care Med 1994; 150:1453-1455.
43. Gimble J, Guilak F. Adipose-derived adult stem cells: Isolation, characterization, and differentiation potential. Cytotherapy 2003; 5:362-369.
44. Aust L, Devlin B, Foster S J, Halvorsen Y D, Hicok K, du Laney T. Sen A, Willingmyre G D, Gimble J M. Yield of human adipose-derived adult stem cells from liposuction aspirates. Cytotherapy 2004; 6:7-14.
45. Lee R H, Pulin A A. Seo M J, Kota D J, Ylostalo J, Larson B L, Semprun-Prieto L, Delafontaine P, Prockop D J. Intravenous hmscs improve myocardial infarction in mice because cells embolized in lung are activated to secrete the anti-inflammatory protein tsg-6. Cell Stem Cell 2009; 5:54-63.
46. Van Eeden S, Yeung A, Quinlam K, Hogg J C. Systemic response to ambient particulate matter relevance to chronic obstructive pulmonary disease. Proc Am Thorac Soc 2005:2:61-67.
47. Shapiro, S. D. Vascular atrophy and VEGFR-2 signaling: old theories of pulmonary emphysema meet new data. J Clin Invest 106, 1309-1310 (2000)

1. A method of treating a patient, comprising the step of:
administering a therapeutically effective dose of adipose stem cell-derived conditioned medium (ASC-CM) to a patient who has been diagnosed with at least one lung injury or disease to treat the lung injury or disease.

2. The method of claim 1, further comprising the steps of:
obtaining a quantity of adipose stem cells (ASCs) from a human; and
culturing at least some of the quantity of adipose stem cells in a culture medium to generate the ASC-CM;
wherein the steps of obtaining and culturing are performed prior to the step of administering the ASC-CM to the patient.

3. The method of claim 2, wherein the human is the patient.

4. The method of claim 1, further comprising the step of:
obtaining a quantity of adipose stem cells (ASCs) from a human;
culturing at least some of the quantity of adipose stem cells in a culture medium; and
concentrating the culture medium to generate the ASC-CM;
wherein the steps of obtaining and culturing are performed prior to the step of administering the ASC-CM to the patient.

5. A method of treating a patient, comprising the step of:
administering a therapeutically effective dose of an adipose stem cell product to a patient who has been diagnosed with at least one lung injury or disease to treat the lung injury or disease.

6. The method of claim 5, further comprising the steps of:
isolating a quantity of adipose stem cells from adipose tissue obtained from a human; and
preparing the adipose stem cell product using at least some of the quantity of adipose stem cells;
wherein the steps of isolating and preparing are performed prior to the step of administering the adipose stem cell product to the patient.

7. The method of claim 6, wherein the human is the patient.

8. The method of claim 5, further comprising the steps of:
isolating a quantity of adipose stem cells from adipose tissue obtained from a human;
culturing at least some of the quantity of adipose stem cells to obtain a culture of adipose stem cells; and
preparing the adipose stem cell product using at least some of the culture of adipose stem cells;
wherein the steps of isolating, culturing, and preparing are performed prior to the step of administering the adipose stem cell product to the patient.

9. The method of claim 8, wherein the human is the patient.

10. The method of claim 5, wherein the adipose stem cell product comprises a quantity of adipose stem cells.

11. The method of claim 5, wherein the adipose stem cell product comprises a conditioned medium obtained by culturing adipose stem cells.

12. A method of treating a patient, comprising the step of:
administering a therapeutically effective dose of a product to a patient who has been diagnosed with at least one lung injury or disease to treat the lung injury or disease, the product selected from the group consisting of adipose stem cells (ASCs) and adipose stem cell-derived conditioned medium (ASC-CM).
